(19) United States
(12) Patent Application Publication
(10) Pub. No.: US 2022/0233249 A1
(43) Pub. Date: Jul. 28, 2022

(54) MEDICAL SYSTEM AND METHOD OF USE

(71) Applicant: Tsunami MedTech, LLC, Menlo Park, CA (US)

(72) Inventor: John H. SHADDUCK, Menlo Park, CA (US)

(73) Assignee: Tsunami MedTech, LLC, Menlo Park, CA (US)

(21) Appl. No.: 17/647,835

(22) Filed: Jan. 12, 2022

Related U.S. Application Data
(60) Provisional application No. 63/136,992, filed on Jan. 13, 2021.

(51) Int. Cl. A61B 34/20; A61B 5/06; A61B 1/273; A61B 1/018; A61B 18/04; A61B 1/05

(52) U.S. Cl. CPC A61B 34/20; A61B 5/066; A61B 1/273; A61B 2034/105; A61B 18/04; A61B 1/05; A61B 1/018

(57) ABSTRACT

Medical instruments, systems, and methods for applying energy to tissue, and more particularly ablating, sealing, coagulating, shrinking or creating lesions in tissue by means of contacting a targeted tissue in a patient with a vapor phase media wherein a subsequent vapor-to-liquid phase change of the media applies thermal energy to the tissue to cause an intended therapeutic effect. Variations include devices and methods for generating a flow of high-quality vapor and monitoring the vapor flow for various parameters with one or more sensors. In yet additional variations, the invention includes devices and methods for modulating parameters of the system in response to the observed parameters.

### RELATED APPLICATION

This is a non-provisional application of U.S. provisional application No. 63/136,992 filed on Jan. 13, 2021, the entirety of which is incorporated by reference herein.

### FIELD OF THE INVENTION

This invention relates to medical instruments and systems for applying energy to tissue and more particularly relates to a system for ablating, sealing, coagulating, shrinking or creating lesions in tissue by means of contacting a targeted tissue in a patient with a vapor phase media wherein a subsequent vapor-to-liquid phase change of the media applies thermal energy to the tissue to cause an intended therapeutic effect. Variations of the invention include devices and methods for generating a flow of high-quality vapor and monitoring the vapor flow for various parameters with one or more sensors. In yet additional variations, the invention includes devices and methods for modulating parameters of the system in response to the observed parameters.

### BACKGROUND OF THE INVENTION

Various types of medical instruments utilizing radiofrequency (RF) energy, laser energy, microwave energy and the like have been developed for delivering thermal energy to tissue, for example, to ablate tissue. While such prior art forms of energy delivery work well for some applications, RF, laser and microwave energy typically cannot cause highly “controlled” and “localized” thermal effects that are desirable in controlled ablation soft tissue for ablating a controlled depth or for the creation of precise lesions in such tissue. In general, the non-linear or non-uniform characteristics of tissue affect electromagnetic energy distributions in tissue.

What is needed are systems and methods that controllably apply thermal energy in a controlled and localized manner without the lack of control often associated when Rf, laser and microwave energy are applied directly to tissue.

### SUMMARY OF THE INVENTION

The present invention is adapted to provide improved methods of controlled thermal energy delivery to localized tissue volumes, for example, for ablating, sealing, coagulating, or otherwise damaging targeted tissue, for example, to ablate a tissue volume interstitially or to ablate the lining of a body cavity. Of particular interest, the method causes thermal effects in targeted tissue without the use of RF current flow through the patient's body and without the potential of carbonizing tissue. The devices and methods of the present disclosure allow the use of such energy modalities to be used as an adjunct rather than a primary source of treatment.

One variation of the present novel method includes a method of delivering energy into a target tissue of a body region, the method comprising advancing a working end of a device into the body region, expanding a structure from within a working end of the device into the body region, where at least a portion of the thin-wall structure is permeable to allow transfer of a medium through the structure to the tissue, and delivering an amount of energy from the structure to treat the target tissue of the body region.

Expanding the structure can include everting the structure. Although the variations described below discuss everting the structure, alternate variations can include inflating, unfolding, unfurling or unrolling the structure. Typically, these different expansion modes relate to the manner in which the structure is located (partially or fully) within the working end of the device. In any case, many variations of the method and device allow for the structure to expand to (or substantially) to the cavity or tissue region being treated. As such, the structure can comprise a thin-wall structure or other structure that allows for delivery of the vapor media therethrough. Expansion of the structure can occur using a fluid or gas. Typically, the expansion pressure is low. However, alternate variations can include the use of high-pressure expansion. In such a variation, the expansion of the structure can be used to perform a therapeutic treatment in conjunction with the energy delivery.

Typically, the energy applied by the vapor media is between 25 W and 150 W. In additional variations, the vapor media can apply a first amount of energy with alternate energy modalities being used to provide additional amounts of energy as required by the particular application or treatment. Such additional energy modalities include RF energy, light energy, radiation, resistive heating, chemical energy and/or microwave energy. In some cases, the treatment ablates the target tissue. In alternate variations, the treatment coagulates or heats the tissue to affect a therapeutic purpose. The additional modalities of energy can be applied from elements that are in the expandable structure or on a surface of the structure.

Turning now to the vapor delivery, as described below, the vapor transfers an amount of energy to the tissue without charring or desiccating the tissue. In certain variations, delivering the amount of energy comprises delivering energy using a vapor media by passing the vapor media through the structure. Accordingly, the expandable structure can include at least one vapor outlet. However, additional variations of the method or device can include structures that include a plurality of permeable portions, where at least a porosity of one of the permeable portions varies such that delivery of the amount of energy is non-uniform about the structure when expanded. In one example, delivering the amount of energy comprises delivering a first amount of energy at a central portion of the structure when expanded and a second amount of energy at a distal or proximal portion, and where the first amount of energy is different than the second amount of energy.

In those variations that employ additional energy delivery means, a second amount of energy can be delivered from a portion of the structure. For example, electrodes, antennas, or emitters, can be positioned on or within the structure.

The structures included within the scope of the methods and devices described herein can include any shape as required by the particular application. Such shapes include, but are not limited to round, non-round, flattened, cylindrical, spiraling, pear-shaped, triangular, rectangular, square, oblong, oblate, elliptical, banana-shaped, donut-shaped, pancake-shaped, or a plurality or combination of such shapes when expanded. The shape can even be selected to conform to a shape of a cavity within the body (e.g., a passage of the esophagus, a chamber of the heart, a portion of the GI tract, the stomach, blood vessel, lung, uterus, cervical canal, fallopian tube, sinus, airway, gall bladder, pancreas, colon, intestine, respiratory tract, etc.)

In additional variations, the devices and methods described herein can include one or more additional expanding members. Such additional expanding members can be positioned at a working end of the device. The second expandable member can include a surface for engaging a non-targeted region to limit the energy from transferring to the non-targeted region. The second expandable member can be insulated to protect the non-targeted region. Alternatively, or in combination, the second expandable member can be expanded using a cooling fluid where the expandable member conducts cooling to the non-targeted region. Clearly, any number of additional expandable members can be used. In one variation, an expandable member can be used to seal an opening of the cavity.

In certain variations, the device or method includes the use of one or more vacuum passages so that upon monitoring a cavity pressure within the cavity, to relieve pressure when the cavity pressure reaches a pre-determined value.

In another variation, a device according to the present disclosure can include an elongated device having an axis and a working end, a vapor source communicating with at least one vapor outlet in the working end, the vapor source providing a condensable vapor through the vapor outlet to contact the targeted tissue region, such that when the condensable vapor contacts the targeted tissue region, an amount of energy transfers from the condensable vapor to the targeted tissue region, and at least one expandable member is carried by the working end, the expandable member having a surface for engaging a non-targeted tissue region to limit contact and energy transfer between the condensable vapor and the non-targeted tissue region.

In one variation, a first and second expandable members are disposed axially proximal of the at least one vapor outlet. This allows treatment distal to the expandable members. In another variation, at least one vapor outlet is intermediate the first and second expandable members. Therefore, the treatment occurs between the expandable members. In yet another variation, at least one expandable member is radially positioned relative to at least one vapor outlet to radially limit the condensable vapor from engaging the non-targeted region.

In additional variations of the methods and devices, the expandable member(s) is fluidly coupled to a fluid source for expanding the expandable member. The fluid source can optionally comprise a cooling fluid that allows the expandable member to cool tissue via conduction through the surface of the expandable member.

In another variation of a method under the principles of the present invention, the method includes selectively treating a target region of tissue and preserving a non-target region of tissue within a body region. For example, the method can include introducing a working end of an axially-extending vapor delivery tool into cavity or lumen, the working end comprising at least one vapor outlet being fluidly coupleable to a vapor source having a supply of vapor, expanding at least one expandable member carried by the working end to engage the non-target region of tissue, and delivering the vapor through the vapor outlet to the target region tissue to cause energy exchange between the vapor and the target region tissue such that vapor contact between the non-target region of tissue is minimized or prevented by the at least one expanding member.

The methods described herein can also include a variation of treating esophageal tissue of a patient's body. In such a case, any of the variations of the devices described herein can be used. In any case, an example of the method includes introducing an elongate vapor delivery tool into an esophageal passage, the vapor delivery tool being coupleable to a supply of vapor, delivering the vapor through the delivery tool into the passage, and controlling energy application to a surface of the passage by controlling interaction between the vapor and the surface of the passage. In an additional variation, the elongate vapor delivery tool includes a vapor lumen and a vacuum lumen, where the vapor lumen and vacuum lumen are in fluid communication, where controlling interaction between the vapor and the surface of the passage comprises modulating delivery of a vapor inflow through the vapor lumen and modulating vacuum outflow through the vacuum lumen. The method can further include applying a cooling media to the surface of the passage to limit diffusion of heat in the surface.

Methods of the present disclosure also include methods of reducing diabetic conditions. For example, the method can include treating a patient to reduce diabetic conditions by inserting a vapor delivery device to a digestive passage, where the vapor delivery device is coupleable to a source of vapor, delivering the vapor to a wall of the digestive tract to transfer energy from the vapor to the wall in a sufficient amount to alter a function of the digestive tract, and controlling interaction between the vapor and the wall to cause controlled ablation at a treatment area. The treatment can be applied in an organ selected from the group consisting of the stomach, the small intestines, the large intestines, and the duodenum. In some variations, controlling interaction between the vapor and the wall causes a thin ablation layer on a surface of the wall.

The present disclosure also includes medical systems for applying thermal energy to tissue, where the system comprises an elongated probe with an axis having an interior flow channel extending to at least one outlet in a probe working end; a source of vapor media configured to provide a vapor flow through at least a portion of the interior flow channel, wherein the vapor has a minimum temperature; and at least one sensor in the flow channel for providing a signal of at least one flow parameter selected from the group one of (i) existence of a flow of the vapor media, (ii) quantification of a flow rate of the vapor media, and (iii) quality of the flow of the vapor media. The medical system can include variations where the minimum temperature varies from at least 80° C., 100° C. 120° C., 140° C. and 160° C. However, other temperature ranges can be included depending upon the desired application.

Sensors included in the above system include a temperature sensor, an impedance sensor, a pressure sensor as well as an optical sensor.

The source of vapor media can include a pressurized source of a liquid media and an energy source for phase conversion of the liquid media to a vapor media. In addition, the medical system can further include a controller capable of modulating a vapor parameter in response to a signal of a flow parameter; the vapor parameter selected from the group of (i) flow rate of pressurized source of liquid media, (ii) inflow pressure of the pressurized source of liquid media, (iii) temperature of the liquid media, (iv) energy applied from the energy source to the liquid media, (v) flow rate of vapor media in the flow channel, (vi) pressure of the vapor media in the flow channel, (vi) temperature of the vapor media, and (vii) quality of vapor media.

In another variation, a novel medical system for applying thermal energy to tissue comprises an elongated probe with an axis having an interior flow channel extending to at least one outlet in a probe working end, wherein a wall of the flow channel includes an insulative portion having a thermal conductivity of less than a maximum thermal conductivity; and a source of vapor media configured to provide a vapor flow through at least a portion of the interior flow channel, wherein the vapor has a minimum temperature.

Variations of such systems include systems where the maximum thermal conductivity ranges from 0.05 W/mK, 0.01 W/mK and 0.005 W/mK.

Methods are disclosed herein for thermally treating tissue by providing a probe body having a flow channel extending therein to an outlet in a working end, introducing a flow of a liquid media through the flow channel and applying energy to the tissue by inductively heating a portion of the probe sufficient to vaporize the flowing media within the flow channel causing pressurized ejection of the media from the outlet to the tissue.

The methods can include applying energy between 10 and 10,000 Joules to the tissue from the media. The rate at which the media flows can be controlled as well.

In another variation, the methods described herein include inductively heating the portion of the probe by applying an electromagnetic energy source to a coil surrounding the flow channel. The electromagnetic energy can also inductively heat a wall portion of the flow channel.

Another variation of the method includes providing a flow permeable structure within the flow channel. Optionally, the coil described herein can heat the flow permeable structure to transfer energy to the flow media. Some examples of a flow permeable structure include woven filaments, braided filaments, knit filaments, metal wool, a microchannel structure, a porous structure, a honeycomb structure, and an open-cell structure. However, any structure that is permeable to flow can be included.

The electromagnetic energy source can include an energy source ranging from a 10-Watt source to a 500-Watt source.

Medical systems for treating tissue are also described herein. Such systems can include a probe body having a flow channel extending therein to an outlet in a working end, a coil about at least a portion or the flow channel, and an electromagnetic energy source coupled to the coil, where the electromagnetic energy source induces current in the coil causing energy delivery to a flowable media in the flow channel. The systems can include a source of flowable media coupled to the flow channel. The electromagnetic energy source can be capable of applying energy to the flowable media sufficient to cause a liquid-to-vapor phase change in at least a portion of the flowable media as described in detail herein. In addition, the probe can include a sensor selected from a temperature sensor, an impedance sensor, a capacitance sensor and a pressure sensor. In some variations, the probe is coupled to an aspiration source.

The medical system can also include a controller capable of modulating at least one operational parameter of the source of flowable media in response to a signal from a sensor. For example, the controller can be capable of modulating a flow of the flowable media. In another variation, the controller is capable of modulating a flow of the flowable media to apply between 100 and 10,000 Joules to the tissue.

The systems described herein can also include a metal portion in the flow channel for contacting the flowable media. The metal portion can be a flow permeable structure and can optionally comprise a microchannel structure. In additional variations, the flow permeable structure can include woven filaments, braided filaments, knit filaments, metal wool, a porous structure, a honeycomb structure, an open-cell structure or a combination thereof.

In another variation, the methods described herein can include positioning a probe in an interface with a targeted tissue and causing a vapor media from to be ejected from the probe into the interface with tissue wherein the media delivers energy ranging from 5 joules to 100,000 joules to cause a therapeutic effect, wherein the vapor media is converted from a liquid media within the probe by inductive heating means.

Methods described herein also include methods of treating tissue by providing medical system including a heat applicator portion for positioning in an interface with targeted tissue, and converting a liquid media into a vapor media within an elongated portion of the medical system having a flow channel communicating with a flow outlet in the heat applicator portion, and contacting the vapor media with the targeted tissue to thereby deliver energy ranging from 5 joules to 100,000 joules to cause a therapeutic effect.

As discussed herein, the methods can include converting the liquid into a vapor media using an inductive heating means. In an alternate variation, a resistive heating means can be combined with the inductive heating means or can replace the inductive heating means.

The instrument and method of the invention can cause an energy-tissue interaction that is imageable with intra-operative ultrasound or MRI.

The instrument and method of the invention cause thermal effects in tissue that do not rely on applying an electrical field across the tissue to be treated.

Additional advantages of the invention will be apparent from the following description, the accompanying drawings, and the appended claims.

All patents, patent applications and publications mentioned in this specification are herein incorporated by reference to the same extent as if each individual publication or patent application was specifically and individually indicated to be incorporated by reference.

In addition, it is intended that combinations of aspects of the systems and methods described herein as well as the various embodiments themselves, where possible, are within the scope of this disclosure.

This application is related to the following U.S Non-provisional and Provisional applications: Application No. 61/126,647 Filed on May 6, 2008 MEDICAL SYSTEM AND METHOD OF USE (docket TSMT-P-T004.20-US); Application No. 61/126,651 Filed on May 6, 2008 MEDICAL SYSTEM AND METHOD OF USE (docket TSMT-P-T004.40-US); TSMT-P-T004.50-U.S. Application No. 61/126,612 Filed on May 6, 2008 MEDICAL SYSTEM AND METHOD OF USE (docket TSMT-P-T004.40-US); Application No. 61/126,636 Filed on May 6, 2008 MEDICAL SYSTEM AND METHOD OF USE (docket TSMT-P-T004.60-US; Application No. 61/130,345 Filed on May 31, 2008 MEDICAL SYSTEM AND METHOD OF USE (docket TSMT-P-T004.70-US); Application No. 61/066,396 Filed on Feb. 20, 2008 TISSUE ABLATION SYSTEM AND METHOD OF USE (TSMT-P-T005.60-US); Application No. 61/123,416 Filed on Apr. 8, 2008 MEDICAL SYSTEM AND METHOD OF USE (TSMT-P-T005.70-US); Application No. 61/068,049 Filed on Mar. 4, 2008 MEDICAL SYSTEM AND METHOD OF USE (TSMT-P-T005.80-US); Application No. 61/123,384 Filed on Apr. 8, 2008 MEDICAL SYSTEM AND METHOD OF USE (TSMT-P-T005.90-US); Application No. 61/068,130 Filed on Mar. 4, 2008 MEDICAL SYSTEM AND METHOD OF USE (docket TSMT-P-T006.00-US); Application No. 61/123,417 Filed on Apr. 8, 2008 MEDICAL SYSTEM AND METHOD OF USE (docket TSMT-P-T006.10-US); Application No. 61/123,412 Filed on Apr. 8, 2008 MEDICAL SYSTEM AND METHOD OF USE (docket TSMT-P-T006.20-US); Application No. 61/126,830 Filed on May 7, 2008 MEDICAL SYSTEM AND METHOD OF USE (docket TSMT-P-T006.40-US); and Application No.: 61/126,620 Filed on May 6, 2008 MEDICAL SYSTEM AND METHOD OF USE (docket TSMT-P-T006.50-US).

The systems and methods described herein are also related to U.S. patent application Ser. No. 10/681,625 filed Oct. 7, 2003 titled “Medical Instruments and Techniques for Thermally-Mediated Therapies”; Ser. No. 11/158,930 filed Jun. 22, 2005 titled “Medical Instruments and Techniques for Treating Pulmonary Disorders”; Ser. No. 11/244,329 (Docket No. S-TT-00200A) filed Oct. 5, 2005 titled “Medical Instruments and Methods of Use” and Ser. No. 11/329,381 (Docket No. S-TT-00300A) filed Jan. 10, 2006 titled “Medical Instrument and Method of Use”.

All of the above applications are incorporated herein by this reference and made a part of this specification, together with the specifications of all other commonly invented applications cited in the above applications.

### BRIEF DESCRIPTION OF THE DRAWINGS

FIG. 1A is a graphical depiction of the quantity of energy needed to achieve the heat of vaporization of water.

FIG. 1B is a diagram of phase change energy release that underlies a system and method of the invention.

FIG. 2 shows a schematic view of a medical system that is adapted for treating a target region of tissue.

FIG. 3 is a block diagram of a control method of the invention.

FIG. 4A is an illustration of the working end of FIG. 2 being introduced into soft tissue to treat a targeted tissue volume.

FIG. 4B is an illustration of the working end of FIG. 4A, showing the propagation of vapor media in tissue in a method of use in ablating a tumor.

FIG. 5 is an illustration of a working end similar to FIGS. 4A-4B with vapor outlets comprising microporosities in a porous wall.

FIG. 6A is a schematic view of a needle-type working end of a vapor delivery tool for applying energy to tissue.

FIG. 6B is a schematic view of an alternative needle-type working end similar to FIG. 6A.

FIG. 6C is a schematic view of a retractable needle-type working end similar to FIG. 6B.

FIG. 6D is a schematic view of working end with multiple shape-memory needles.

FIG. 6E is a schematic view of a working end with deflectable needles.

FIG. 6F is a schematic view of a working end with a rotating element for directing vapor flows.

FIG. 6G is another view of the working end of FIG. 6F.

FIG. 6H is a schematic view of a working end with a balloon.

FIG. 6I is a schematic view of an articulating working end.

FIG. 6J is a schematic view of an alternative working end with RF electrodes.

FIG. 6K is a schematic view of an alternative working end with a resistive heating element.

FIG. 6L is a schematic view of a working end with a tissue-capturing loop.

FIG. 6M is a schematic view of an alternative working end with jaws for capturing and delivering vapor to tissue.

FIG. 7 is a schematic view of an alternative working end for delivering vapor to tissue.

FIG. 8 is a schematic view of an alternative working end for delivering vapor to tissue.

FIG. 9 is a partly disassembled view of a handle and inductive vapor generator system of the invention.

FIG. 10 is an enlarged schematic view of the inductive vapor generator of FIG. 9.

FIG. 11A is a sectional view of the working end of a vapor delivery tool comprising an introducer carrying an expandable structure for delivering vapor from outlets therein.

FIG. 11B is a view of the structure of FIG. 11A depicting an initial step of a method of expanding the thin-wall structure in a body cavity.

FIG. 11C is a sectional view of a structure of FIG. 11B in a deployed, expanded configuration depicting the step of delivering vapor into tissue surrounding the body cavity.

FIG. 12A is a schematic view of the handle and working end of vapor delivery tool for treating an esophageal disorder such as Barrett's esophagus.

FIG. 12B is another view of the vapor delivery tool of FIG. 12A illustrating an initial step of a method of the invention comprising expanding proximal and distal occlusion balloons to define a treatment site between the balloons.

FIG. 12C is a view similar to that of FIG. 12B illustrating a subsequent step of expanding one or more additional occlusion balloons to further circumscribe the targeted treatment site and the step of delivering vapor to ablate the esophageal lumen.

FIG. 13 is a view similar to that of FIGS. 12A-12C illustrating an alternative embodiment and method for using a scalloped balloon for providing a less than 360° ablation the esophageal lumen.

FIG. 14 depicts an alternative method for accomplishing a local ablation within the esophageal lumen utilizing an elongated vapor delivery tool introduced through a working channel of an endoscope.

FIG. 15 is a sectional view of the working end of the vapor delivery tool of FIG. 14, showing vapor outlets that cooperates with an aspiration lumen for local control of vapor contact with tissue.

FIG. 16 is an illustration of a catheter with two spaced-apart occlusion balloons introduced into a patient's colon to treat the disorder therein.

FIG. 17 is an enlarged view of the working end of the catheter of FIG. 16.

FIG. 18 is a cross-sectional view of the catheter shaft of FIG. 17 taken along line 18-18 of FIG. 17.

FIG. 19 is a partially cut-away view of another catheter working end with first and second occlusion balloons having different wall thicknesses.

FIG. 20 is another catheter working end with first and second occlusion balloons wherein the treatment space between the spaced-apart balloons can be adjusted in axial of length with a telescoping member.

FIG. 21 is a schematic view of an alternative system for vapor treatment of a body lumen wherein the treatment catheter with a plurality of occlusion balloons is introduced through an endoscope.

FIG. 22 is an enlarged view of the working end of the catheter FIG. 21.

FIG. 23 is a cross-sectional view of the catheter shaft of FIG. 22 taken along line 23-23 of FIG. 22.

FIG. 24A is a schematic illustration of an initial step of a method of the invention using the catheter of FIG. 22 in ablating a thin layer of the wall of the body lumen.

FIG. 24B is an illustration of a subsequent step of the method of the invention.

FIG. 24C is an illustration of a subsequent step of the method of the invention.

FIG. 24D is an illustration of a subsequent step of the method of the invention.

FIG. 24E is an illustration of a subsequent step of the method of the invention.

FIG. 25 is a schematic view of the working end of the catheter with an electrical contact sensor carried in the surface of at least one occlusion balloon.

FIG. 26 is a schematic view of another variation of the catheter with occlusion balloons wherein at least one balloon carries an electrical sensor for determining suitable contact between the balloon in the wall of the lumen.

FIG. 27 illustrates another variation of the invention where a single-use probe integrates an image sensor with a treatment catheter as described above.

FIG. 28A is a view of the distal end of the probe of FIG. 27 with a working end of the treatment catheter in a non-extended or retracted position.

FIG. 28B is a view of the distal end of the probe of FIG. 27 with the working end of the treatment catheter in an extended position.

FIG. 29A is a view of a subject's stomach, duodenum, pancreas, and liver showing the introduction of an endoscope into the duodenum and the advancement of a treatment catheter through the working channel in the endoscope, and more particularly illustrating the first, second, third and fourth parts of the duodenum.

FIG. 29B is a view of the subject's duodenum as in FIG. 29A with the endoscope positioned to view of the major and minor duodenal papilla and showing the method of introducing the treatment catheter to a position where a first occlusion balloon is adjacent to the major and minor duodenal papilla in preparation to ablate duodenal mucosa.

FIG. 29C illustrates another step in a method of the invention wherein a first occlusion balloon is expanded to cover and occlude the major and minor duodenal papilla.

FIG. 29D illustrates a subsequent step in the method of the invention wherein a second distal occlusion balloon is expanded, and the controller and vapor source are operated to deliver vapor from the catheter shaft to the space between the first and second occlusion balloons to ablate the duodenal mucosa.

FIG. 30 is a view of a subject's duodenum and the use of the catheter having first, second, and third occlusion balloons that allow for ablation of the duodenal mucosa both proximally and distally from the occlusion balloon covering the major and minor duodenal papilla.

FIG. 31 is a view of a subject's duodenum and the use of the catheter similar to that of FIGS. 29A-29D with an elongated space between the first and second occlusion balloons.

FIG. 32A is a view of a subject's duodenum and a catheter similar to that of FIG. 31 with a pair of intermediate occlusion balloons in the elongated space between the first and second occlusion balloons.

FIG. 32B is a view of the catheter of FIG. 32A with a distal intermediate occlusion balloon inflated to split the axial length of the elongated treatment space between the first and second occlusion balloons further showing delivering vapor to ablate mucosa in a proximal space.

FIG. 32C is a view of the catheter of FIG. 32B with the proximal intermediate occlusion balloon inflated to treat a distal space between the first and second occlusion balloons.

FIG. 33 illustrates another variation of the invention that comprises an imaging system that provides a virtual image of a portion a patient's intestinal tract, including the duodenum that is adapted for treatment planning as well as for controlling a location of the endoscope and treatment catheter for performing a mucosal ablation.

FIG. 34 illustrates a conventional endoscope and a component of the invention comprising an elastomeric sleeve carrying a sensor component to cooperate with a sensing system to determine the spatial location of the endoscope to generate a virtual image thereof.

FIG. 35A schematically illustrates the introduction of the endoscope of FIG. 34 into a patient's duodenum with a sensor system spatially locating the endoscope to provide a virtual image thereof.

FIG. 35B illustrates the continued advancement of the endoscope as in FIG. 35A in the patient's duodenum and advancement of a delivery catheter outwardly from the endoscope with a sensor system spatially locating the catheter distal end to provide a virtual image thereof.

FIG. 36A depicts virtual images of (i) a portion of the intestinal tract, (ii) the endoscope, and (iii) the treatment catheter on a display screen.

FIG. 36B depicts the virtual images of FIG. 36A with the treatment catheter in a different position

FIG. 37 schematically illustrates a robotic system configured to introduce an endoscope into a patient's duodenum and to advance a treatment catheter.

FIG. 38 illustrates a touch screen display adapted to operate a robotic system to navigate an endoscope and a treatment catheter in a patient's small intestine.

FIG. 39A illustrates a first step in a method of the invention to create a virtual image of a patient's duodenum where a sensor system cooperates with a sensor component carried by the endoscope to spatially map its location.

FIG. 39B illustrates a subsequent step in the method of FIG. 39A where the sensor system spatially maps another location of the endoscope.

FIG. 39C illustrates another subsequent step in the method of FIGS. 39A-39B where the sensor system spatially maps yet another location of the endoscope.

FIG. 40 illustrates a virtual image of the patient's duodenum generated from the spatial location data provided by the sensor system when tracking movement of the catheter as shown in FIGS. 39A-39C.

### DETAILED DESCRIPTION OF THE INVENTION

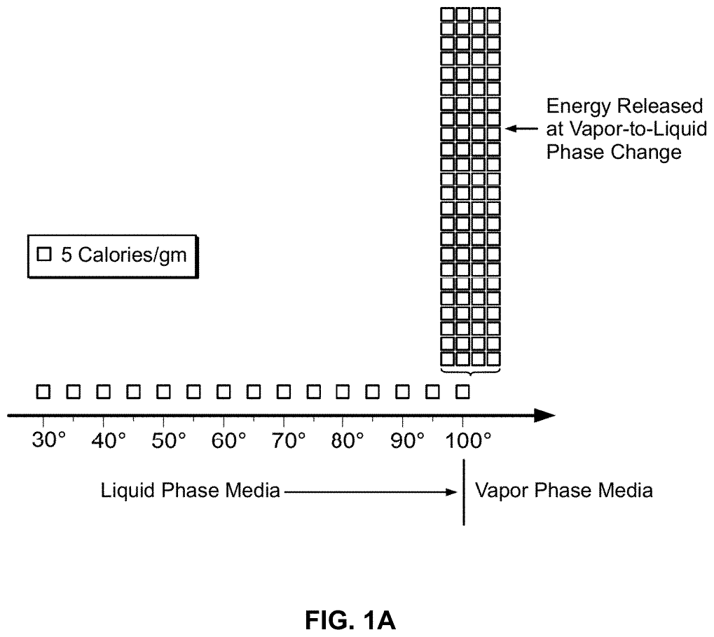

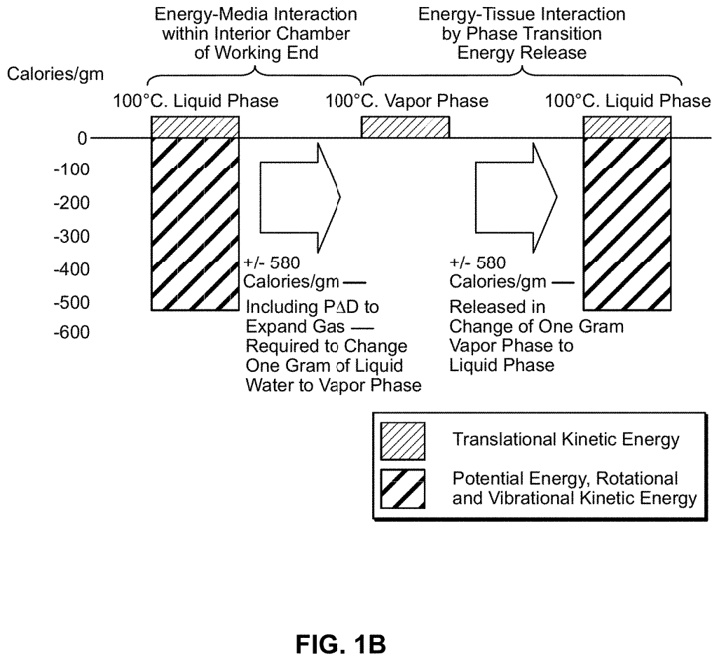

In general, the thermally mediated treatment method comprises causing a vapor-to-liquid phase state change in a selected media at a targeted tissue site, thereby applying thermal energy substantially equal to the heat of vaporization of the selected media to the tissue site. The thermally mediated therapy can be delivered to tissue by such vapor-to-liquid phase transitions, or “internal energy” releases, about the working surfaces of several types of instruments for ablative treatments of soft tissue. FIGS. 1A and 1B illustrate the phenomena of phase transitional releases of internal energies. Such internal energy involves energy on the molecular and atomic scale—and in polyatomic gases is directly related to intermolecular attractive forces, as well as rotational and vibrational kinetic energy. In other words, the method of the invention exploits the phenomenon of internal energy transitions between gaseous and liquid phases that involve very large amounts of energy compared to specific heat.

It has been found that the controlled application of such energy in a controlled media-tissue interaction solves many of the vexing problems associated with energy-tissue interactions in RF, laser, and ultrasound modalities. The apparatus of the invention provides a vaporization chamber in the interior of an instrument, in an instrument's working end, or in a source remote from the instrument end. A source provides liquid media to the interior vaporization chamber, wherein energy is applied to create a selected volume of vapor media. In the process of the liquid-to-vapor phase transition of a liquid media, for example, water, large amounts of energy are added to overcome the cohesive forces between molecules in the liquid, and an additional amount of energy is required to expand the liquid 1000+ percent (PAD) into a resulting vapor phase (see FIG. 1A). Conversely, in the vapor-to-liquid transition, such energy will be released at the phase transition at the interface with the targeted tissue site. That is, the heat of vaporization is released at the interface when the media transitions from gaseous phase to liquid phase, wherein the random, disordered motion of molecules in the vapor regain cohesion to convert to a liquid media. This release of energy (defined as the capacity for doing work) relating to intermolecular attractive forces is transformed into therapeutic heat for a thermotherapy at the interface with the targeted body structure. Heat flow and work are both ways of transferring energy.

In FIG. 1A, the simplified visualization of internal energy is useful for understanding phase transition phenomena that involve internal energy transitions between liquid and vapor phases. If heat were added at a constant rate in FIG. 1A (graphically represented as 5 calories/gm blocks) to elevate the temperature of water through its phase change to a vapor phase, the additional energy required to achieve the phase change (latent heat of vaporization) is represented by the large number of 110+ blocks of energy at 100° C. in FIG. 1A. Still referring to FIG. 1A, it can be easily understood that all other prior art ablation modalities—RF, laser, microwave, and ultrasound—create energy densities by simply ramping up calories/gm as indicated by the temperature range from 37° C. through 100° C. as in FIG. 1A. The prior art modalities make no use of the phenomenon of phase transition energies as depicted in FIG. 1A.

FIG. 1B graphically represents a block diagram relating to energy delivery aspects of the present invention. The system provides for insulative containment of an initial primary energy-media interaction within an interior vaporization chamber of medical thermotherapy system. The initial, ascendant energy-media interaction delivers energy sufficient to achieve the heat of vaporization of a selected liquid media, such as water or saline solution, within an interior of the system. This aspect of the technology requires a highly controlled energy source wherein a computer controller may need to modulated energy application between very large energy densities to initially surpass the latent heat of vaporization with some energy sources (e.g. a resistive heat source, an RF energy source, a light energy source, a microwave energy source, an ultrasound source and/or an inductive heat source) and potential subsequent lesser energy densities for maintaining a high vapor quality. Additionally, a controller must control the pressure or flow rate of liquid flows for replenishing the selected liquid media at the required rate and optionally for controlling propagation velocity of the vapor phase media from the working end surface of the instrument. In use, the method of the invention comprises the controlled application of energy to achieve the heat of vaporization as in FIG. 1A and the controlled vapor-to-liquid phase transition and vapor exit pressure to thereby control the interaction of a selected volume of vapor at the interface with tissue. The vapor-to-liquid phase transition can deposit 400, 500, 600 or more cal/gram within the targeted tissue site to perform the thermal ablation with the vapor in typical pressures and temperatures.

Treatment Liquid Source, Energy Source, Controller

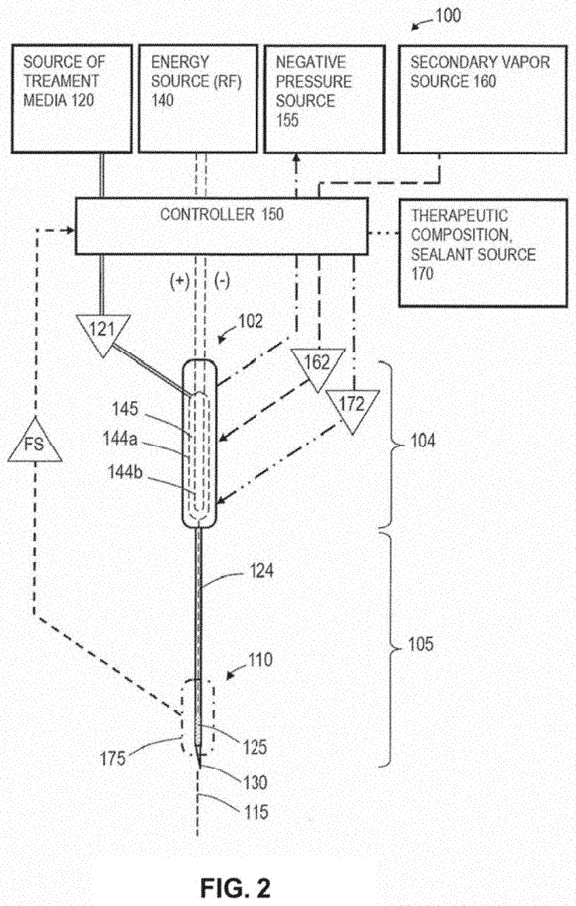

Referring to FIG. 2, a schematic view of medical system 100 of the present invention is shown that is adapted for treating a tissue target, wherein the treatment comprises an ablation or thermotherapy, and the tissue target can comprise any mammalian soft tissue to be ablated, sealed, contracted, coagulated, damaged or treated to elicit an immune response. The system 100 includes an instrument or probe body 102 with a proximal handle end 104 and an extension portion 105 having a distal or working end indicated at 110. In one embodiment depicted in FIG. 2, the handle end 104 and extension portion 105 generally extend about longitudinal axis 115. In the embodiment of FIG. 2, the extension portion 105 is a substantially rigid tubular member with at least one flow channel therein, but the scope of the invention encompasses extension portions 105 of any mean diameter and any axial length, rigid or flexible, suited for treating a particular tissue target. In one embodiment, a rigid extension portion 105 can comprise a 20 Ga. to 40 Ga. needle with a short length for thermal treatment of a patient's cornea or a somewhat longer length for treating a patient's retina. In another embodiment, an elongate extension portion 105 of a vapor delivery tool can comprise a single needle or a plurality of needles having suitable lengths for tumor or soft tissue ablation in a liver, breast, gall bladder, prostate, bone, and the like. In another embodiment, an elongate extension portion 105 can comprise a flexible catheter for introduction through a body lumen to access at tissue target, with a diameter ranging from about 1 to 10 mm. In another embodiment, the extension portion 105 or working end 110 can be articulatable, deflectable or deformable. The probe handle end 104 can be configured as a hand-held member or can be configured for coupling to a robotic surgical system. In another embodiment, the working end 110 carries an openable and closeable structure for capturing tissue between first and second tissue-engaging surfaces, which can comprise actuatable components such as one or more clamps, jaws, loops, snares and the like. The proximal handle end 104 of the probe can carry various actuator mechanisms known in the art for actuating components of the system 100, and/or one or more footswitches can be used for actuating components of the system.

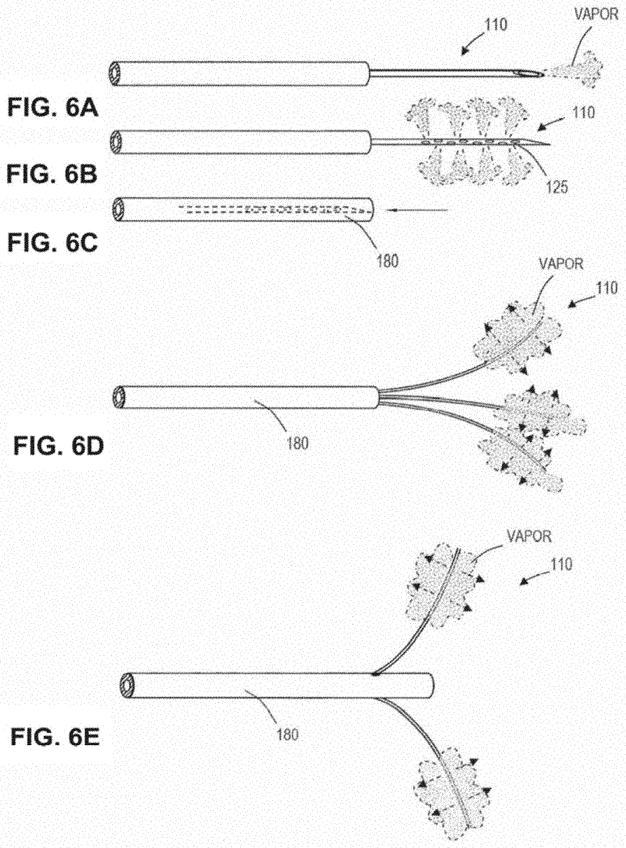

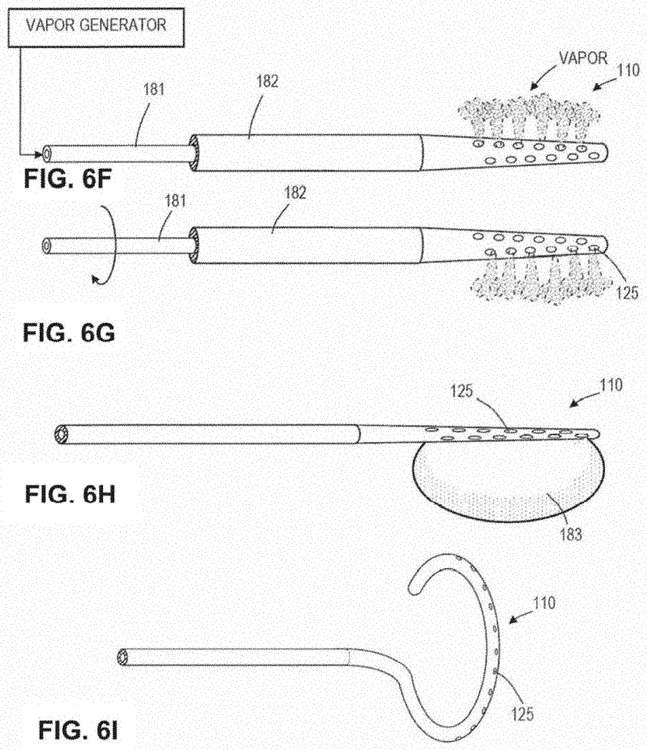

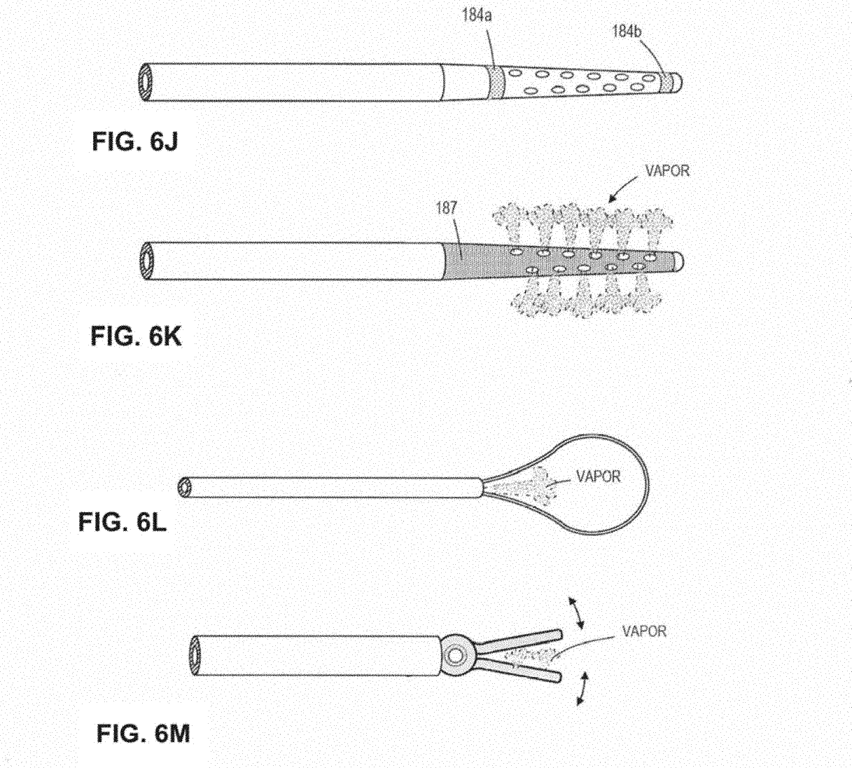

As can be seen in FIG. 2, the system 100 further includes a source 120 of a flowable liquid treatment media 121 that communicates with a flow channel 124 extending through the probe body 102 to at least one outlet 125 in the working end 110. The outlet 125 can be singular or multiple and have any suitable dimension and orientation as will be described further below. The distal tip 130 of the probe can be sharp for penetrating tissue or can be blunt-tipped or open-ended with outlet 125. Alternatively, the working end 110 can be configured in any of the various embodiments shown in FIGS. 6A-6M and described further below.

In one embodiment shown in FIG. 2, an RF energy source 140 is operatively connected to a thermal energy source or emitter (e.g., opposing polarity electrodes 144a, 144b) in interior chamber 145 in the proximal handle end 104 of the probe for converting the liquid treatment media 121 from a liquid phase media to a non-liquid vapor phase media 122 with a heat of vaporization in the range of 60° C. to 200° C., or 80° C. to 120° C. A vaporization system using Rf energy and opposing polarity electrodes is disclosed in co-pending U.S. patent application Ser. No. 11/329,381 which is incorporated herein by reference. Another embodiment of vapor generation system is described in below in the Section titled “INDUCTIVE VAPOR GENERATION SYSTEMS”. In any system embodiment, for example, in the system of FIG. 2, a controller 150 is provided that comprises a computer control system configured for controlling the operating parameters of inflows of liquid treatment media source 120 and energy applied to the liquid media by an energy source to cause the liquid-to-vapor conversion. The vapor generation systems described herein can consistently produce a high-quality vapor having a temperature of at least 80° C., 100° C. 120° C., 140° C. and 160° C.

As can be seen in FIG. 2, the medical system 100 can further include a negative pressure or aspiration source indicated at 155 that is in fluid communication with a flow channel in probe 102 and working end 110 for aspirating treatment vapor media 122, body fluids, ablation by-products, tissue debris and the like from a targeted treatment site, as will be further described below. In FIG. 2, the controller 150 also is capable of modulating the operating parameters of the negative pressure source 155 to extract vapor media 122 from the treatment site or from the interior of the working end 110 by means of a recirculation channel to control flows of vapor media 122 as will be described further below.

In another embodiment, still referring to FIG. 2, medical system 100 further includes secondary media source 160 for providing an inflow of a second media, for example, a biocompatible gas such as CO2. In one method, a second media that includes at least one of depressurized CO2, N2, O2 or H2O can be introduced and combined with the vapor media 122. This second media 162 is introduced into the flow of non-ionized vapor media for lowering the mass average temperature of the combined flow for treating tissue. In another embodiment, the medical system 100 includes a source 170 of a therapeutic or pharmacological agent or a sealant composition indicated at 172 for providing an additional treatment effect in the target tissue. In FIG. 2, the controller indicated at 150 also is configured to modulate the operating parameters of source 160 and 170 to control inflows of a secondary vapor 162 and therapeutic agents, sealants or other compositions indicated at 172.

In FIG. 2, it is further illustrated that a sensor system 175 is carried within the probe 102 for monitoring a parameter of the vapor media 122 to thereby provide a feedback signal FS to the controller 150 by means of feedback circuitry to thereby allow the controller to modulate the output or operating parameters of treatment media source 120, energy source 140, negative pressure source 155, secondary media source 160 and therapeutic agent source 170. The sensor system 175 is further described below, and in one embodiment, comprises a flow sensor to determine flows or the lack of a vapor flow. In another embodiment, the sensor system 175 includes a temperature sensor. In another embodiment, sensor system 175 includes a pressure sensor. In another embodiment, the sensor system 175 includes a sensor arrangement for determining the quality of the vapor media, e.g., in terms of vapor saturation or the like. The sensor systems will be described in more detail below.

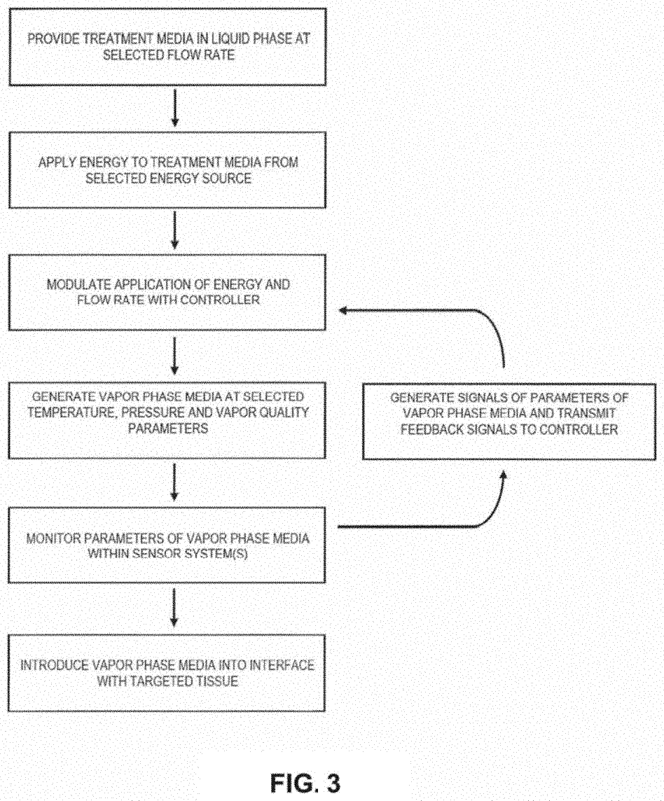

Now turning to FIGS. 2 and 3, the controller 150 is capable of all operational parameters of system 100, including modulating the operational parameters in response to preset values or in response to feedback signals FS from sensor system(s) 175 within the system 100 and probe working end 110. In one embodiment, as depicted in the block diagram of FIG. 3, the system 100 and controller 150 are capable of providing or modulating an operational parameter comprising a flow rate of liquid phase treatment media 122 from pressurized source 120, wherein the flow rate is within a range from about 0.001 to 20 ml/min, 0.010 to 10 ml/min or 0.050 to 5 ml/min. The system 100 and controller 150 are further capable of providing or modulating another operational parameter comprising the inflow pressure of liquid phase treatment media 121 in a range from 0.5 to 1000 psi, 5 to 500 psi, or 25 to 200 psi. The system 100 and controller 150 are further capable of providing or modulating another operational parameter comprising a selected level of energy capable of converting the liquid phase media into a non-liquid, non-ionized gas phase media, wherein the energy level is within a range of about 5 to 2,500 watts; 10 to 1,000 watts or 25 to 500 watts. The system 100 and controller 150 are capable of applying the selected level of energy to provide the phase conversion in the treatment media over an interval ranging from 0.1 second to 10 minutes; 0.5 seconds to 5 minutes, and 1 second to 60 seconds. The system 100 and controller 150 are further capable of controlling parameters of the vapor phase media including the flow rate of non-ionized vapor media proximate an outlet 125, the pressure of vapor media 122 at the outlet, the temperature or mass average temperature of the vapor media, and the quality of vapor media as will be described further below.

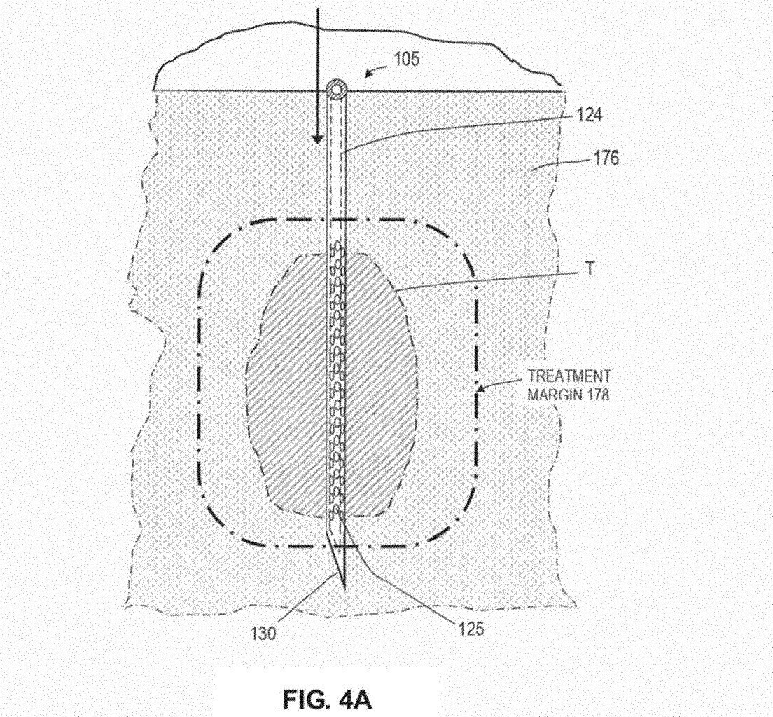

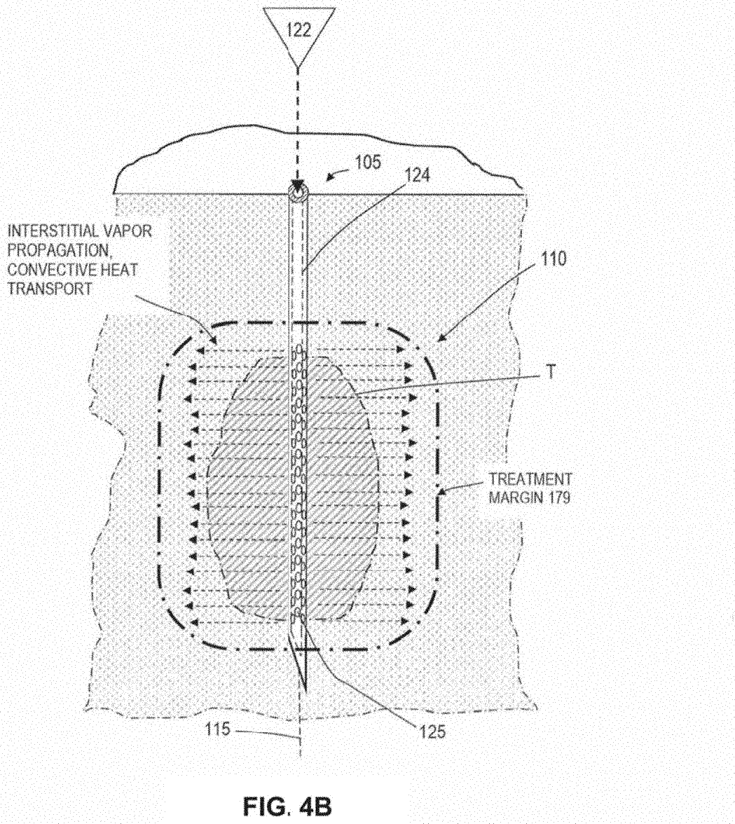

FIGS. 4A and 4B illustrate a working end 110 of the system 100 of FIG. 2 and a method of use. As can be seen in FIG. 4A, a working end 110 is singular and configured as a needle-like device for penetrating into and/or through a targeted tissue T such as a tumor in a tissue volume 176. The tumor can be benign, malignant, hyperplastic or hypertrophic tissue, for example, in a patient's breast, uterus, lung, liver, kidney, gall bladder, stomach, pancreas, colon, GI tract, bladder, prostate, bone, vertebra, eye, brain or other tissue. In one embodiment of the invention, the extension portion 105 is made of a metal, for example, stainless steel. Alternatively, or additionally, at least some portions of the extension portion can be fabricated of a polymer material such as PEEK, PTFE, Nylon or polypropylene. Also, optionally, one or more components of the extension portion are formed of coated metal, for example, a coating with Teflon® to reduce friction upon insertion and to prevent tissue sticking following use. In one embodiment at in FIG. 4A, the working end 110 includes a plurality of outlets 125 that allow vapor media to be ejected in all radial directions over a selected treatment length of the working end. In another embodiment, the plurality of outlets can be symmetric or asymmetric axially or angularly about the working end 110.

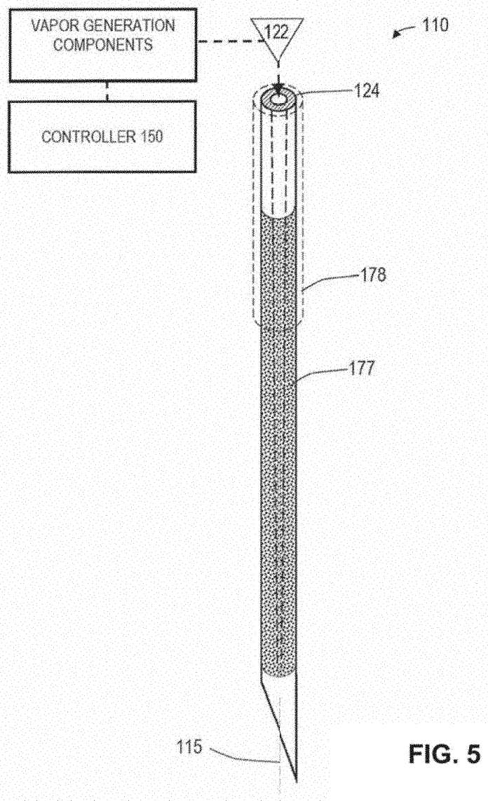

In one embodiment, the outer diameter of extension portion 105 or working end 110 is, for example, 0.2 mm, 0.5 mm, 1 mm, 2 mm, 5 mm or an intermediate, smaller, or larger diameter. Optionally, the outlets can comprise microporosities 177 in a porous material as illustrated in FIG. 5 for diffusion and distribution of vapor media flows about the surface of the working end. In one such embodiment, such porosities provide a greater restriction to vapor media outflows than adjacent targeted tissue, which can vary greatly in vapor permeability. In this case, such microporosities ensure that vapor media outflows will occur substantially uniformly over the surface of the working end. Optionally, the wall thickness of the working end 110 is from 0.05 to 0.5 mm. Optionally, the wall thickness decreases or increases towards the distal sharp tip 130 (FIG. 5). In one embodiment, the dimensions and orientations of outlets 125 are selected to diffuse and/or direct vapor media propagation into targeted tissue T and more particularly to direct vapor media into all targeted tissue to cause extracellular vapor propagation and thus convective heating of the target tissue as indicated in FIG. 4B. As shown in FIGS. 4A-4B, the shape of the outlets 125 can vary, for example, round, ellipsoid, rectangular, radially and/or axially symmetric or asymmetric. As shown in FIG. 5, a sleeve 178 can be advanced or retracted relative to the outlets 125 to provide a selected exposure of such outlets to provide vapor injection over a selected length of the working end 110. Optionally, the outlets can be oriented in various ways, for example, so that vapor media 122 is ejected perpendicular to a surface of working end 110 or ejected is at an angle relative to the axis 115 or angled relative to a plane perpendicular to the axis. Optionally, the outlets can be disposed on a selected side or within a selected axial portion of the working end, wherein rotation or axial movement of the working end will direct vapor propagation and energy delivery in a selected direction. In another embodiment, the working end 110 can be disposed in a secondary outer sleeve that has apertures in a particular side thereof for angular/axial movement in targeted tissue for directing vapor flows into the tissue.

FIG. 4B illustrates the working end 110 of system 100 ejecting vapor media from the working end under selected operating parameters, for example, a selected pressure, vapor temperature, vapor quantity, vapor quality and duration of flow. The duration of flow can be a selected preset or the hyperechoic aspect of the vapor flow can be imaged by means of ultrasound to allow the termination of vapor flows by observation of the vapor plume relative to targeted tissue T. As depicted schematically in FIG. 4B, the vapor can propagate extracellularly in soft tissue to provide intense convective heating as the vapor collapses into water droplets, which results in effective tissue ablation and cell death. As further depicted in FIG. 4B, the tissue is treated to provide an effective treatment margin 179 around a targeted tumorous volume. The vapor delivery step is continuous or can be repeated at a high repetition rate to cause a pulsed form of convective heating and thermal energy delivery to the targeted tissue. The repetition rate vapor flows can vary, for example, with flow duration intervals from 0.01 to 20 seconds and intermediate off intervals from 0.01 to 5 seconds or intermediate, larger or smaller intervals.

In an exemplary embodiment as shown in FIGS. 4A-4B, the extension portion 105 can be a unitary member such as a needle. In another embodiment, the extension portion 105 or working end 110 can be a detachable flexible body or rigid body, for example, of any type selected by a user with outlet sizes and orientations for a particular procedure with the working end attached by threads or Luer fitting to a more proximal portion of probe 102.

In other embodiments, the working end 110 can comprise needles with terminal outlets or side outlets as shown in FIGS. 6A-6B. The needle of FIGS. 6A and 6B can comprise a retractable needle, as shown in FIG. 6C, capable of retraction into probe or sheath 180 for navigation of the probe through a body passageway or for blocking a portion of the vapor outlets 125 to control the geometry of the vapor-tissue interface. In another embodiment shown in FIG. 6D, the working end 110 can have multiple retractable needles that are of a shape memory material. In another embodiment as depicted in FIG. 6E, the working end 110 can have at least one deflectable and retractable needle that deflects relative to an axis of the probe 180 when advanced from the probe. In another embodiment, the working end 110 as shown in FIGS. 6F-6G can comprise a dual sleeve assembly wherein vapor-carrying inner sleeve 181 rotates within outer sleeve 182 and wherein outlets in the inner sleeve 181 only register with outlets 125 in outer sleeve 182 at selected angles of relative rotation to allow vapor to exit the outlets. This assembly thus provides for a method of pulsed vapor application from outlets in the working end. The rotation can be from about 1 rpm to 1000 rpm.

In another embodiment of FIG. 6H, the working end 110 has a heat applicator surface with at least one vapor outlet 125 and at least one expandable member 183, such as a balloon for positioning the heat applicator surface against targeted tissue. In another embodiment of FIG. 6I, the working end can be a flexible material that is deflectable by a pull-wire as is known in the art. The embodiments of FIGS. 6H and 61 have configurations for use in treating atrial fibrillation, for example, in pulmonary vein ablation.

In another embodiment of FIG. 6J, the working end 110 includes additional optional heat applicator means, which can comprise a mono-polar electrode cooperating with a ground pad or bi-polar electrodes 184a and 184b for applying energy to tissue. In FIG. 6K, the working end 110 includes resistive heating element 187 for applying energy to tissue. FIG. 6L depicts a snare for capturing tissue to be treated with vapor and FIG. 6M illustrates a clamp or jaw structure. The working end 110 of FIG. 6M includes means actuatable from the handle for operating the jaws.

Sensors for Vapor Flows, Temperature, Pressure, Quality

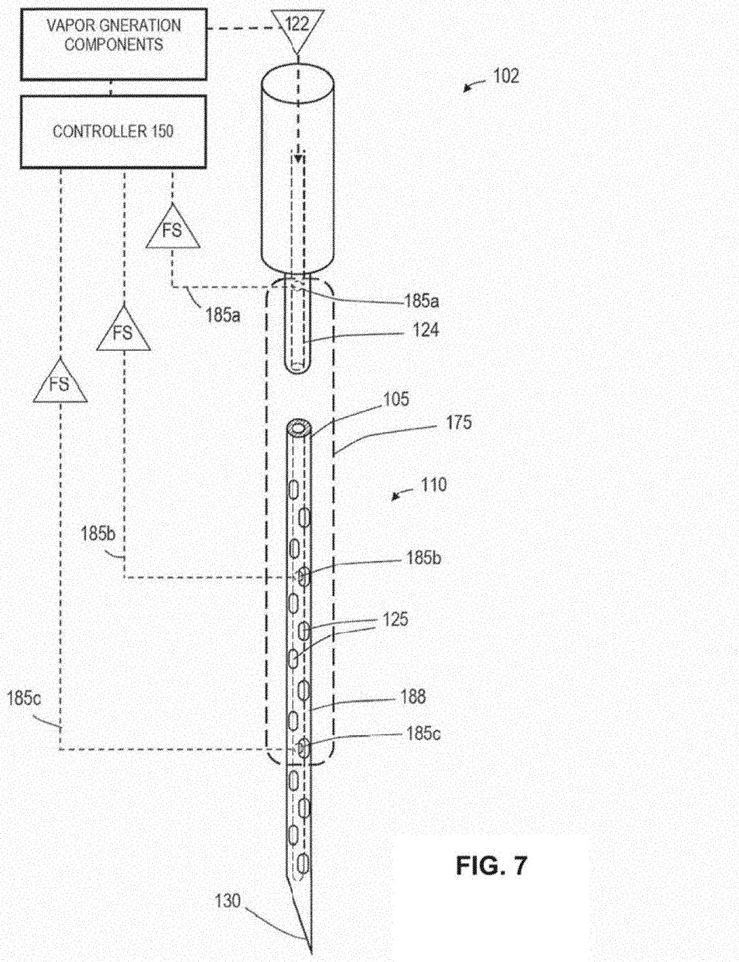

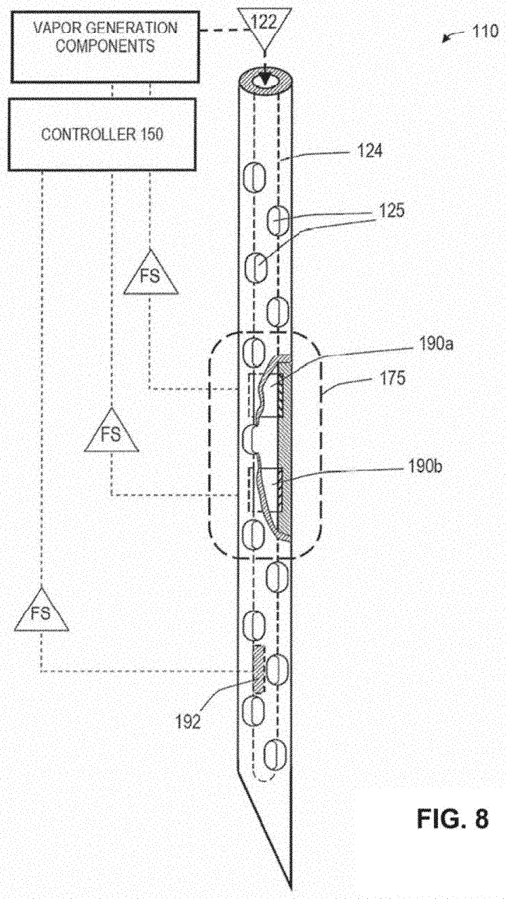

Referring to FIG. 7, one embodiment of sensor system 175 is shown that is carried by working end 110 of the probe 102 depicted in FIG. 2 for determining a first vapor media flow parameter, which can consist of determining whether the vapor flow is in an “on” or “off” operating mode. The working end 110 of FIG. 7 comprises a sharp-tipped needle suited for needle ablation of any neoplasia or tumor tissue, such as a benign or malignant tumor as described previously but can also be any other form of vapor delivery tool. The needle can be any suitable gauge, and in one embodiment, has a plurality of vapor outlets 125. In a typical treatment of targeted tissue, it is important to provide a sensor and feedback signal indicating whether there is a flow, or leakage, of vapor media 122 following treatment or in advance of treatment when the system is in “off” mode. Similarly, it is important to provide a feedback signal indicating a flow of vapor media 122 when the system is in “on” mode. In the embodiment of FIG. 7, the sensor comprises at least one thermocouple or other temperature sensor indicated at 185a, 185b and 185c that are coupled to leads (indicated schematically at 186a, 186b and 186c) for sending feedback signals to controller 150. The temperature sensor can be a singular component or can be a plurality of components spaced-apart over any selected portion of the probe and working end. In one embodiment, a feedback signal of any selected temperature from any thermocouple in the range of the heat of vaporization of treatment media 122 would indicate that flow of vapor media, or the lack of such a signal would indicate the lack of a flow of vapor media. The sensors can be spaced-apart by at least 0.05 mm, 1 mm, 5 mm, 10 mm and 50 mm. In other embodiments, multiple temperature sensing event can be averaged over time, averaged between spaced-apart sensors, the rate of change of temperatures can be measured and the like. In one embodiment, the leads 186a, 186b and 186c are carried in an insulative layer of wall 188 of the extension member 105. The insulative layer of wall 188 can include any suitable polymer or ceramic for providing thermal insulation. In one embodiment, the exterior of the working end also is also provided with a lubricious material, such as Teflon®, which further insures against any tissue sticking to the working end 110.

Still referring to FIG. 7, a sensor system 175 can provide a different type of feedback signal FS to indicate a flow rate or vapor media based on a plurality of temperature sensors spaced-apart within flow channel 124. In one embodiment, the controller 150 includes algorithms capable of receiving feedback signals FS from at least first and second thermocouples (e.g., 185a and 185c) at very high data acquisition speeds and compare the difference in temperatures at the spaced-apart locations. The measured temperature difference, when further combined with the time interval following the initiation of vapor media flows, can be compared against a library to thereby indicate the flow rate.

Another embodiment of sensor system 175 in a similar working end 110 is depicted in FIG. 8, wherein the sensor is configured for indicating vapor quality—in this case, based on a plurality of spaced-apart electrodes 190a and 190b coupled to controller 150 and an electrical source (not shown). In this embodiment, a current flow is provided within a circuit to the spaced-apart electrodes 190a and 190b and during vapor flows within channel 124 the impedance will vary depending on the vapor quality or saturation, which can be processed by algorithms in controller 150 and can be compared to a library of impedance levels, flow rates and the like to thereby determine vapor quality. It is important to have a sensor to provide feedback of vapor quality, which determines how much energy is being carried by a vapor flow. The term “vapor quality” is herein used to describe the percentage of the flow that is actually water vapor as opposed to water droplets that is not phase-changed. In another embodiment (not shown), an optical sensor can be used to determine vapor quality, wherein a light emitter and receiver can determine vapor quality based on transmissibility or reflectance of light relative to a vapor flow.

FIG. 8 further depicts a pressure sensor 192 in the working end 110 for providing a signal as to vapor pressure. In operation, the controller can receive the feedback signals FS relating to temperature, pressure, and vapor quality to thereby modulate all other operating parameters described above to optimize flow parameters for a particular treatment of a target tissue, as depicted in FIG. 1. In one embodiment, a MEMS pressure transducer is used, which are known in the art. In another embodiment, a MEMS accelerometer coupled to a slightly translatable coating can be utilized to generate a signal of changes in flow rate, or a MEMS microphone can be used to compare against a library of acoustic vibrations to generate a signal of flow rates.

Inductive Vapor Generation Systems

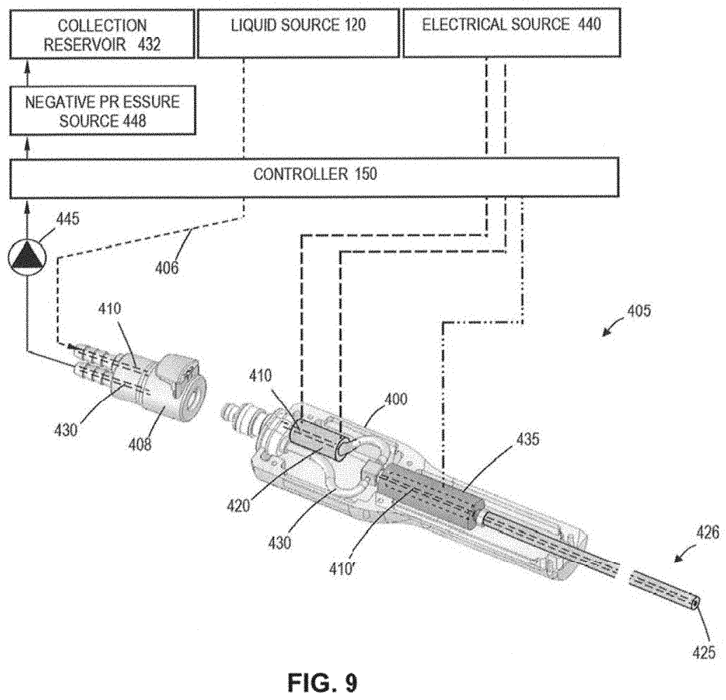

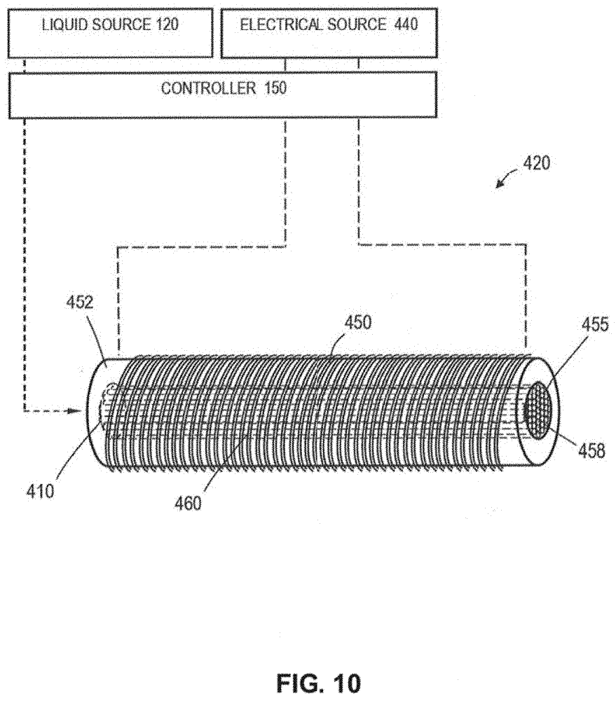

FIGS. 9 and 10 depict a vapor generation component that utilizes and an inductive heating system within a handle portion 400 of the probe or vapor delivery tool 405. In FIG. 9, it can be seen that a pressurized source of liquid media 120 (e.g., water or saline) is coupled by conduit 406 to a quick-connect fitting 408 to deliver liquid into a flow channel 410 extending through an inductive heater 420 in probe handle 400 to at least one outlet 425 in the working end 426. In one embodiment shown in FIG. 9, the flow channel 410 has a bypass or recirculation channel portion 430 in the handle or working end 426 that can direct vapor flows to a collection reservoir 432. In operation, a valve 435 in the flow channel 410 thus can direct vapor generated by inductive heater 420 to either flow channel portion 410′ or the recirculation channel portion 430. In the embodiment of FIG. 10, the recirculation channel portion 430 also is a part of the quick-connect fitting 408.

In FIG. 9, it can be seen that the system includes a computer controller 150 that controls (i) the electromagnetic energy source 440 coupled to inductive heater 420, (ii) the valve 435, which can be an electrically operated solenoid, (iii) an optional valve 445 in the recirculation channel 430 that can operate in unison with valve 435, and (iv) optional negative pressure source 448 operatively coupled to the recirculation channel 430.

In general, the system of the invention provides a small handheld device including an assembly that utilizes electromagnetic induction to turn sterile water flow into superheated or dry vapor, which is propagated from at least one outlet in a vapor delivery tool to interface with tissue, and thus ablate tissue. In one aspect of the invention, an electrically conducting microchannel structure or other flow-permeable structure is provided, and an inductive coil causes electric current flows in the structure. Eddies within the current create magnetic fields, and the magnetic fields oppose the change of the main field, thus raising electrical resistance and resulting in instant heating of the microchannel or other flow-permeable structure. In another aspect of the invention, it has been found that corrosion-resistant microtubes of low magnetic 316 SS are best suited for the application, or a sintered microchannel structure of similar material. While magnetic materials can improve the induction heating of a metal because of ferromagnetic hysteresis, such magnetic materials (e.g. carbon steel) are susceptible to corrosion and are not optimal for generating vapor used to ablate tissue. In certain embodiments, the electromagnetic energy source 440 is adapted for inductive heating of a microchannel structure with a frequency in the range of 50 kHz to 2 Mhz, and more preferably in the range of 400 kHz to 500 kHz. While a microchannel structure is described in more detail below, it should be appreciated that the scope of the invention includes flow-permeable conductive structures selected from the group of woven filaments structures, braided filament structures, knit filaments structures, metal wool structures, porous structures, honeycomb structures and an open-cell structure.

In general, a method of the invention comprises utilizing an inductive heater 420 of FIGS. 9-10 to instantly vaporize a treatment media such as deionized water that is injected into the heater at a flow rate of ranging from 0.001 to 20 ml/min, 0.010 to 10 ml/min, 0.050 to 5 ml/min., and to eject the resulting vapor into body structure to ablate tissue. The method further comprises providing an inductive heater 420 configured for a disposable had-held device (see FIG. 9) that is capable of generating a minimum water vapor that is at least 70% water vapor, 80% water vapor and 90% water vapor.

FIG. 10 is an enlarged schematic view of inductive heater 420 which includes at least one winding of inductive coil 450 wound about an insulative sleeve 452. The coil 450 is typically wound about a rigid insulative member, but also can comprise a plurality of rigid coil portions about a flexible insulator or a flexible coil about a flexible insulative sleeve. The coil can be in a handle portion of a probe or in a working end of a probe such as a catheter. The inductive coil can extend in length at least 5 mm, 10 mm, 25 mm, 50 mm or 100 m.

In one embodiment shown schematically in FIG. 10, the inductive heater 420 has a flow channel 410 in the center of insulative sleeve 452, wherein the flows pass through an inductively heatable microchannel structure indicated at 455. The microchannel structure 455 comprises an assembly of metal hypotubes 458, for example, consisting of thin-wall biocompatible stainless-steel tube tightly packed in bore 460 of the assembly. The coil 450 can thereby inductively heat the metal walls of the microchannel structure 455 and the very large surface area of structure 455 in contact with the flow can instantly vaporize the flowable media pushed into the flow channel 410. In one embodiment, a ceramic insulative sleeve 452 has a length of 1.5″ and outer diameter of 0.25″ with a 0.104″ diameter bore 460 therein. A total of thirty-two 316 stainless steel tubes 458 with 0.016″ O.D., 0.010″ I.D., and 0.003″ wall are disposed in bore 460. The coil 450 has a length of 1.0″ and comprises a single winding of 0.026″ diameter tin-coated copper strand wire (optionally with ceramic or Teflon® insulation) and can be wound in a machined helical groove in the insulative sleeve 452. A 200 W RF power source 440 is used operating at 400 kHz with a pure sine wave. A pressurized sterile water source 120 comprises a computer-controlled syringe that provides fluid flows of deionized water at a rate of 3 ml/min, which can be instantly vaporized by the inductive heater 420. At the vapor exit outlet or outlets 125 in a working end, it has been found that various pressures are needed for various tissues and body cavities for optimal ablations, ranging from about 0.1 to 20 psi for ablating body cavities or lumens and about 1 psi to 100 psi for interstitial ablations.

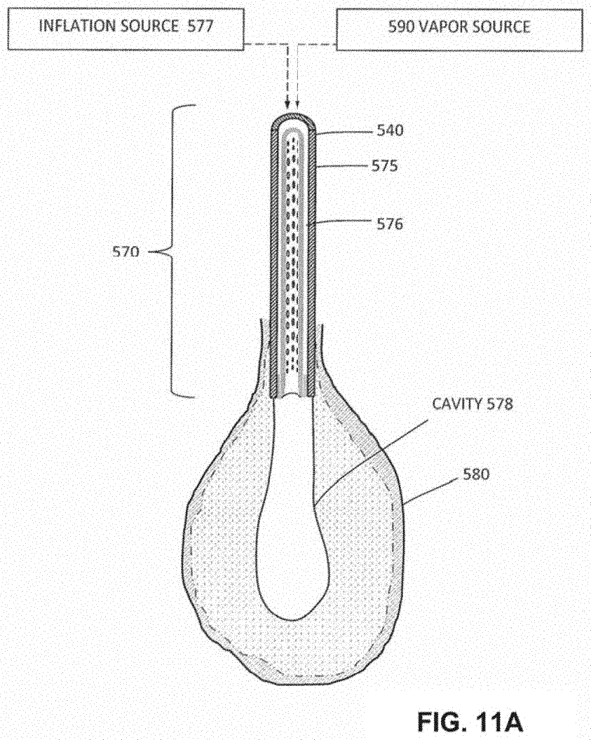

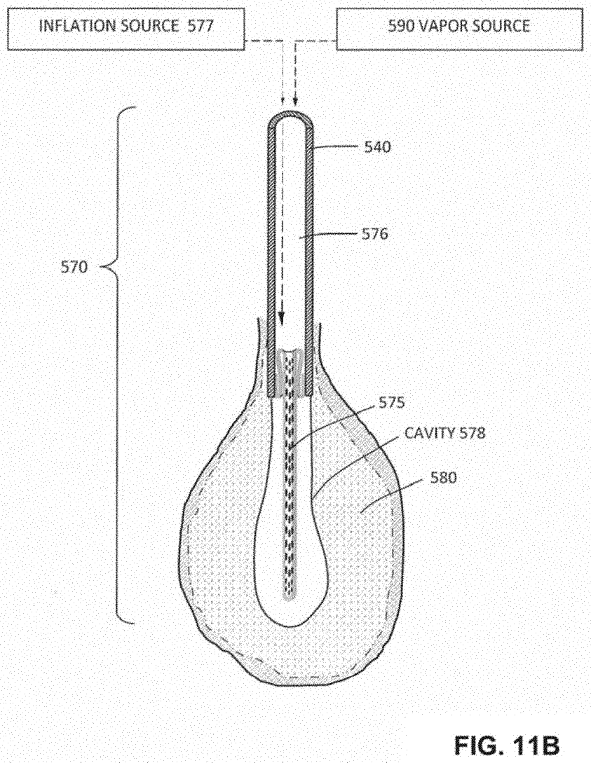

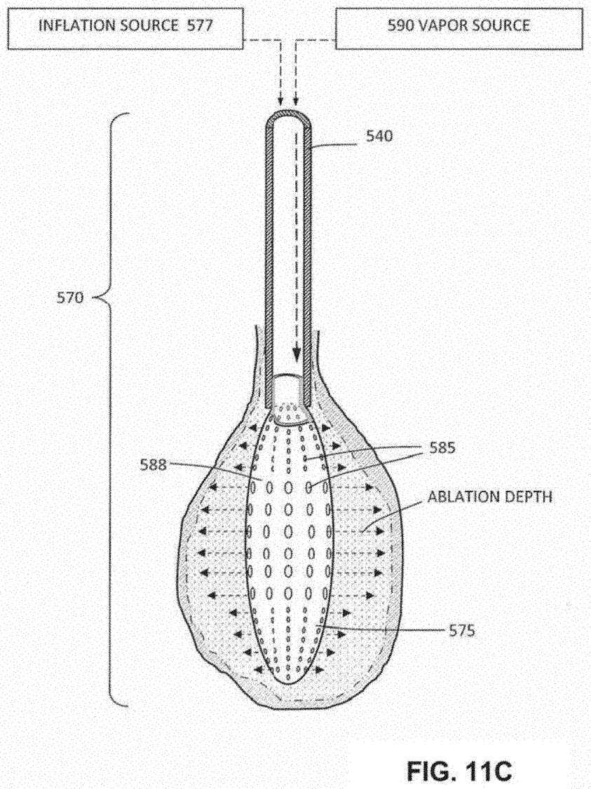

Now turning to FIGS. 11A-11C, a working end that operates similarly to that of FIG. 2 is shown. This embodiment comprises an extension member or other device 540 that can be positioned within a body region as shown in FIG. 11A. The device 540 includes a working end 570 that carries an evertable expansion structure or balloon 575 in interior bore 576. The expansion structure or balloon 575 is everted from within the device into the body region to apply energy to target tissue in the region as described below. By employing via everting, the structure 575 can fill or conform to a desired area within target region. In variations of the device, an everting balloon 575 can be fully positioned within the device 540 prior to everting. Alternatively, the everting balloon 575 can partially extend from an opening in the device 540 and then everted. FIGS. 11B-11C illustrate the balloon 575 being everted by application of fluid generated pressure from a first fluid source 577 (which can be any low-pressure gas in a syringe) within a body cavity 578, for example, a cavity in gall bladder 580. However, additional variations of devices within this disclosure can employ any number of means to evert the balloon 575 from the device 540.

The region containing the target tissue includes any space, cavity, passage, opening, lumen or potential space in a body such as a sinus, airway, blood vessel, uterus, joint capsule, GI tract lumen or respiratory tract lumen. As can be seen in FIG. 11C, the expandable structure 575 can include a plurality of different dimension vapor outlets 585, for locally controlling the ejection pressure of a volume of ejected condensable vapor, which in turn can control the depth and extent of the vapor-tissue interaction and the corresponding depth of ablation. In embodiments described further below, the energy-emitting wall or surface 588 of the expandable structure can carry RF electrodes for applying additional energy to tissue. Light energy emitters or microwave emitters also can be carried by the expandable structure. A vapor flow from source 590 or from any vapor generator source described above can flow high-quality vapor from the vapor ports 585 in the wall or surface 588. The vapor outlets can be dimensioned from about 0.001″ in diameter to about 0.05″ and also can be allowed to be altered in diameter under selected pressures and flow rates. The modulus of a polymer wall 588 also can be selected to control vapor flows through the wall.

In general, a method of the invention as in FIG. 11C for treating a body cavity or luminal tissue comprises (a) everting and/or unfurling a thin-wall structure into the body cavity or lumen, and (b) applying at least 25 W, 50 W, 75 W, 100 W, 125 W and 150 W from an energy-emitter surface of the structure to the tissue, for example, the endometrium for ablation thereof in a global endometrial ablation procedure. In one embodiment, the method applies energy that is provided by a condensable vapor undergoing a phase change. In one embodiment, the method delivers a condensable vapor that provides energy of at least 250 cal/gm, 300 cal/gm, 350 cal/gm, 400 cal/gm and 450 cal/gm. Also, the method can apply energy provided by at least one of a phase change energy release, light energy, RF energy and microwave energy.

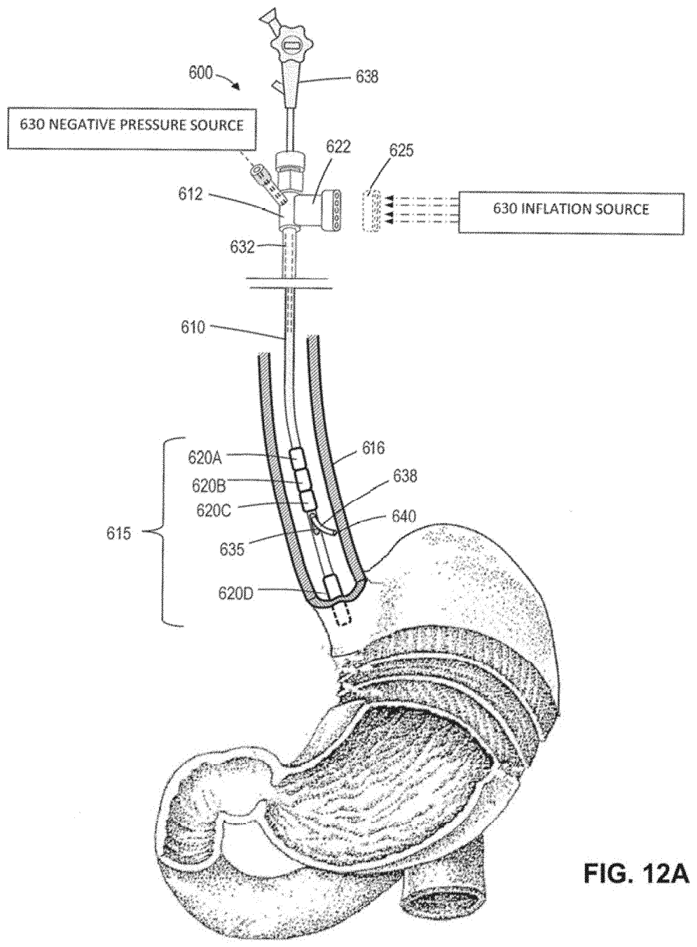

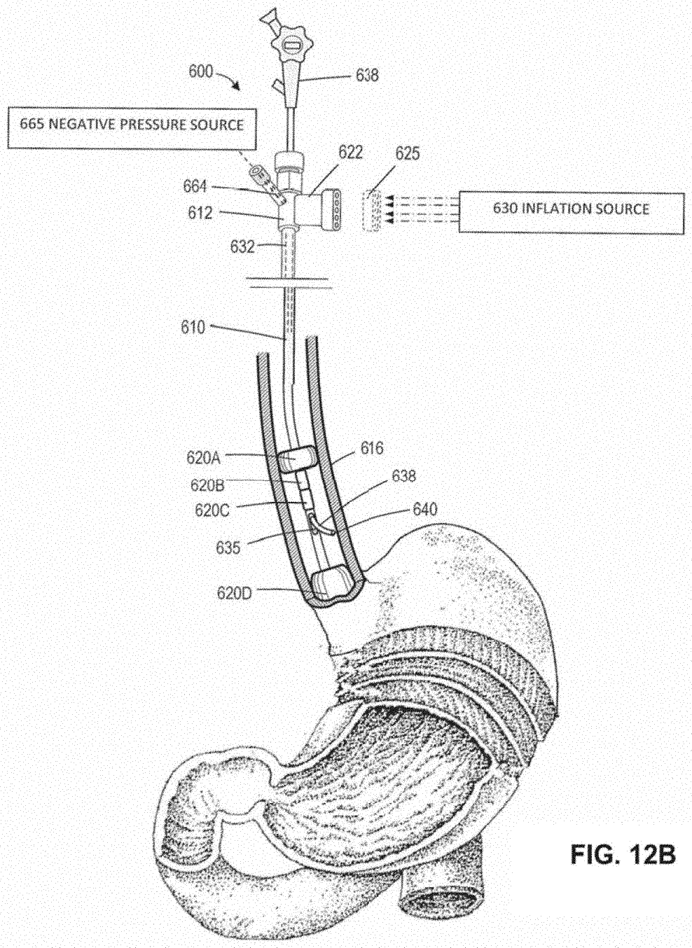

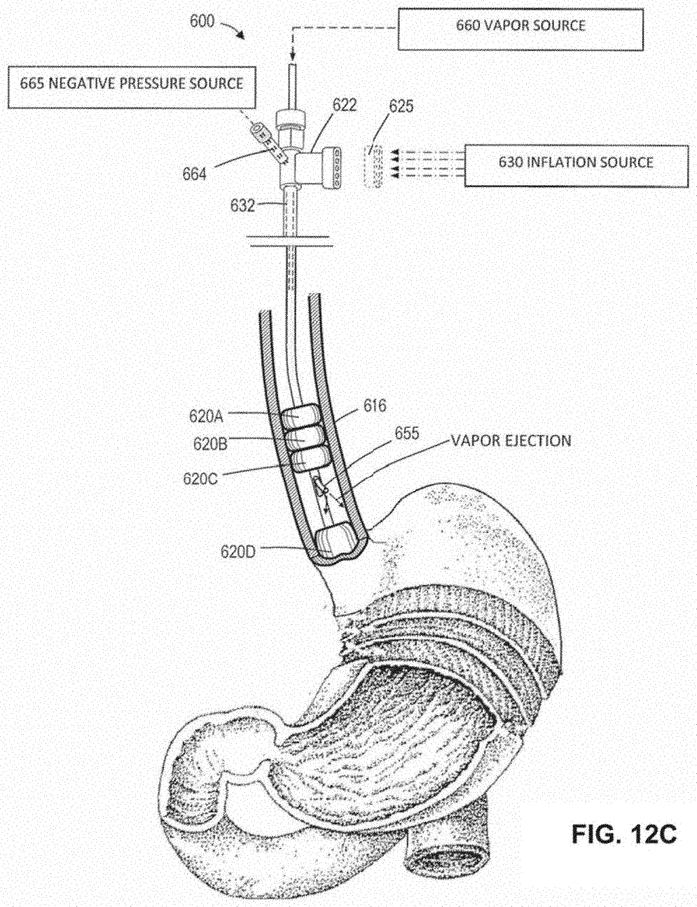

FIGS. 12A-12C depict another embodiment of vapor delivery system 600 that is configured for treating esophageal disorders, such as Barrett's esophagus, dysplasia, esophageal varices, tumors and the like. The objective of a treatment of an esophageal disorder is to ablate a thin layer of the lining of the esophagus, for example, from about 0.1 mm to 1.0 mm in depth. Barrett's esophagus is a severe complication of chronic gastroesophageal reflux disease (GERD) and seems to be a precursor to adenocarcinoma of the esophagus. The incidence of adenocarcinoma of the esophagus due to Barrett's esophagus and GERD is on the rise. In one method of the invention, vapor delivery can be used to ablate a thin surface layer including abnormal cells to prevent the progression of Barrett's esophagus.

The elongated catheter or extension member 610 has a first end or handle end 612 that is coupled to extension member 610 that extends to working end 615. The extension member 610 has a diameter and length suitable for either a nasal or oral introduction into the esophagus 616. The working end 615 of the extension member is configured with a plurality of expandable structures such as temperature resistant occlusion balloons 620A, 620B, 620C and 620D. In one embodiment, the balloons can be compliant silicone. In other embodiment, the balloons can be non-compliant thin-film structures. The handle end 612 includes a manifold 622 that couples to multiple lumens to a connector 625 that allows for each balloon 620A, 620B, 620C and 620D to be expanded independently, for example, with a gas or liquid inflation source indicated at 630. The inflation source 630 can be a plurality of syringes, or a controller can be provided to automatically pump a fluid to selected balloons. The number of balloons carried by extension member 610 can range from 2 to 10 or more. As can be understood in FIGS. 12A-12C, the extension member 610 has independent lumens that communicate with interior chambers of balloons 620A, 620B, 620C and 620D.

Still referring to FIG. 12A, the handle and extension member 610 have a passageway 632 therein that extends to an opening 635 or window to allow a flexible endoscope 638 to view the lining of the esophagus. In one method, a viewing means 640 comprises a CCD at the end of endoscope 638 that can be used to view an esophageal disorder such as Barrett's esophagus in the lower esophagus as depicted in FIG. 12A. The assembly of the endoscope 638 and extension member 610 can be rotated and translated axially, as well as by articulation of the endoscope's distal end. Following the step of viewing the esophagus, the distal balloon 620D can be expanded as shown in FIG. 12B. In one example, the distal balloon 620D is expanded just distal to esophageal tissue targeted for ablative treatment with a condensable vapor. Next, the proximal balloon 620A can be expanded as also shown in FIG. 12B. Thereafter, the targeted treatment area of the esophageal lining can be viewed and additional occlusion balloons 620B and 620C can be expanded to reduce the targeted treatment area. It should be appreciated that the use of occlusion balloons 620A-620D are configured to control the axial length of a vapor ablation treatment, with the thin layer ablation occurring in 360o around the esophageal lumen. In another embodiment, the plurality of expandable members can include balloons that expand to engage only a radial portion of the esophageal lumen for example, 90°, 180° or 270° of the lumen. By this means of utilizing occlusion balloons of a particular shape or shapes, a targeted treatment zone of any axial and radial dimension can be created. One advantage of energy delivery from a phase change is that the ablation will be uniform over the tissue surface that is not contacted by the balloon structures.

FIG. 12C illustrates the vapor delivery step of the method, wherein a high-temperature water vapor is introduced through the extension member 610 and into the esophageal lumen to release energy as the vapor condenses. In FIG. 12C, the vapor is introduced through an elongated catheter 650 that is configured with a distal end 655 that is extendable slightly outside port 635 in the extension member 610. A vapor source 660, such as the vapor generator of FIG. 9 is coupled to the handle end 612 of the catheter. The catheter distal end 655 can have a recirculating vapor flow system as disclosed in commonly invented and co-pending application Ser. No. 12/167,155 filed Jul. 2, 2008. In another embodiment, a vapor source 660 can be coupled directly to a port and lumen 664 at the handle end 612 of extension member 610 to deliver vapor directly through passageway 632 and outwardly from port 635 to treat tissue. In another embodiment, a dedicated lumen in extension member 610 can be provided to allow contemporaneous vapor delivery and use of the viewing means 640 described previously.

The method can include the delivery of vapor for less than 30 seconds, less than 20 seconds, less than 10 seconds or less than 5 seconds to accomplish the ablation. The vapor quality as described above can be greater than 70%, 80%, or 90% and can uniformly ablate the surface of the esophageal lining to a depth of up to 1.0 mm.

In another optional aspect of the invention, also shown in FIGS. 12A-12C, the extension member 610 can include a lumen, for example, the lumen indicated at 664, that can serve as a pressure relief passageway. Alternatively, a slight aspiration force can be applied to the lumen pressure relief lumen from negative pressure source 665.

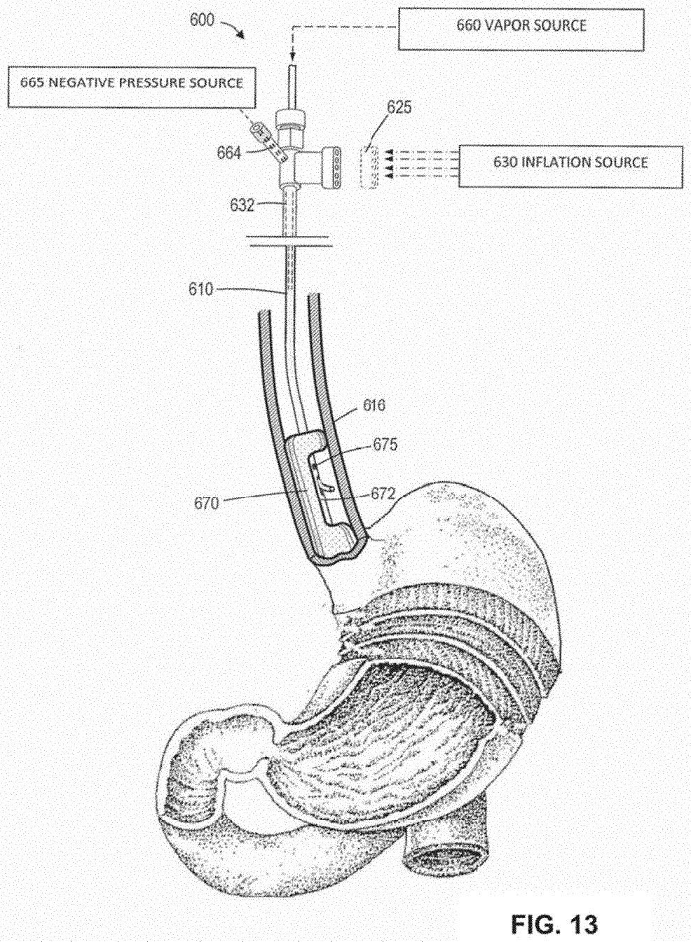

FIG. 13 illustrates another aspect of the invention wherein a single balloon 670 can be configured with a scalloped portion 672 for ablating tissue along one side of the esophageal lumen without a 360-degree ablation of the esophageal lumen. In this illustration, the expandable member or balloon 670 is radially positioned relative to at least one vapor outlet 675 to radially limit the condensable vapor from engaging the non-targeted region. As shown, the balloon 670 is radially adjacent to the vapor outlet 675 so that the non-targeted region of tissue is circumferentially adjacent to the targeted region of tissue. Although the scalloped portion 672 allows radial spacing, alternative designs include one or more shaped balloons or balloons that deploy to a side of the port 675. FIG. 13 also depicts an endoscope 638 extended outward from port 635 to view the targeted treatment region as the balloon 670 is expanded. The balloon 670 can include internal constraining webs to maintain the desired shape. The vapor again can be delivered through a vapor delivery tool or through a dedicated lumen and vapor outlet 675 as described previously. In a commercialization method, a library of catheters can be provided that have balloons configured for a series of less-than-360° ablations of different lengths.

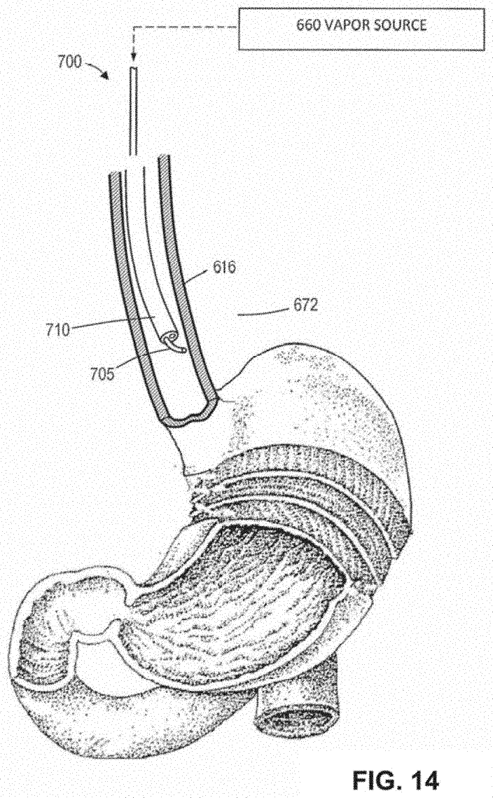

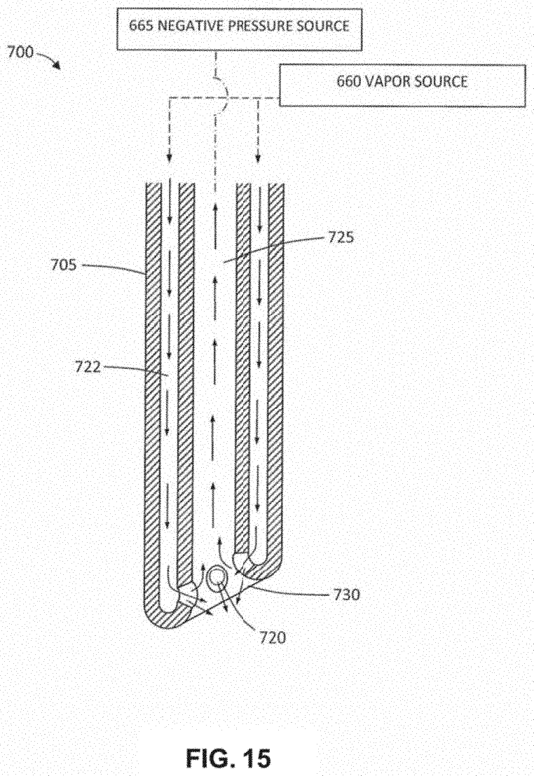

FIGS. 14-15 illustrate another embodiment and method of the invention that can be used for tumor ablation, varices, or Barrett's esophagus in which occlusion balloons are not used. An elongate vapor delivery catheter 700 is introduced along with viewing means to locally ablate tissue. In FIG. 14, catheter 700 having working end 705 is introduced through the working channel of gastroscope 710. Vapor is expelled from the working end 705 to ablate tissue under direct visualization. FIG. 15 depicts a cut-away view of one embodiment of working end in which vapor from source 660 is expelled from vapor outlets 720 in communication with interior annular vapor delivery lumen 722 to contact and ablate tissue. Contemporaneously, the negative pressure source 665 is coupled to central aspiration lumen 725 and is utilized to suction vapor flows back into the working end 705. The modulation of vapor inflow pressure and negative pressure in lumen 725 thus allows precise control of the vapor-tissue contact and ablation. In the embodiment of FIG. 15, the working end can be fabricated of a transparent heat-resistant plastic or glass to allow better visualization of the ablation. In the embodiment of FIG. 15, the distal tip 730 is angled, but it should be appreciated that the tip can be square cut or have any angle relative to the axis of the catheter. The method and apparatus for treating esophageal tissue depicted in FIGS. 14-15 can be used to treat small regions of tissue or can be used in follow-up procedures after an ablation accomplished using the methods and systems of FIGS. 12A-13.

In any of the above methods, a cooling media can be applied to the treated esophageal surface, which can limit the diffusion of heat in the tissue. Besides a cryogenic spray, any cooling liquid such as cold water or saline can be used.

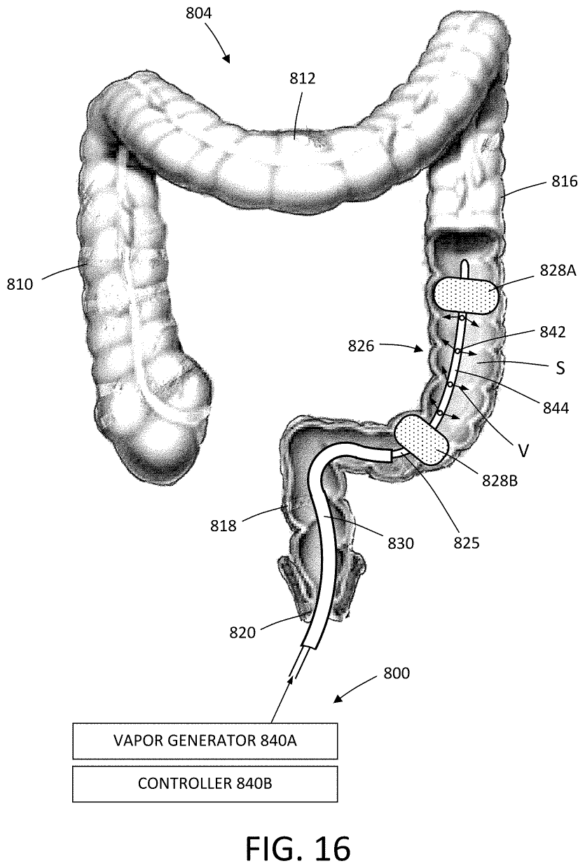

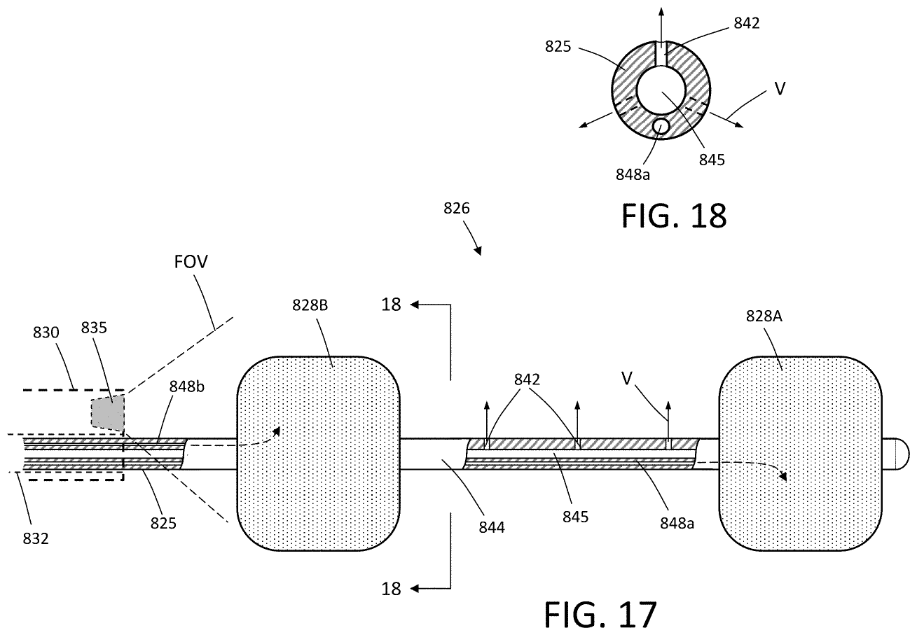

FIGS. 16-17 illustrate another embodiment of vapor delivery system 800 that is configured for delivering ablative energy to a patient's colon to treat a disorder therein, for example, chronic constipation, which is a very common disorder. The role of the colon is to absorb water and to deliver stool to the rectum from where it can be evacuated in a comfortable fashion. Constipation typically arises from disorders of transit through the colon but in other cases can result from disorders of evacuation from the rectum through the anus. The colon 804, also called the large intestine, is shown in FIG. 16. The ileum is the last part of the small intestine and connects to the cecum (first part of the colon) in the lower right abdomen. The remainder of the colon is divided into four segments: the ascending colon 810 travels up the right side of the abdomen, the transverse colon 812 runs across the abdomen, the descending colon 816 travels down the left abdomen, and the sigmoid colon 818 is a short curving of the colon leading to the rectum 820. A key function of the colon is to absorb and remove water, salt, and some nutrients from contents of the colon, thus forming stool. Muscles line the colon's walls and are adapted to squeeze and move its contents through the colon. It should be appreciated that the system 800 also can be adapted to treat other disorders such as irritable bowel syndrome, C. difficile colitis, diverticulitis, Crohn's colitis, ulcerative colitis, infectious colitis, collagenous colitis, lymphocytic colitis, microscopic colitis, flatulence, and metabolic disease. Also, the system 800 can be used to modify or ablate a subject's microbiome, which is known to play a role in many disorders.

As can be understood from FIGS. 16 and 17, a method corresponding to the invention is shown which includes delivering ablative energy from vapor V as described above to ablate a thin interior surface layer of the colon. The surface layers of the colon include passageways for absorbing fluids, and the ablation can modify targeted tissue layers to constrict, damage, seal, or close absorption pathways in the colon wall, which will then provide a greater amount of fluid in the colon, which is retained by the colon contents and which will thus facilitate transit of such contents through the colon.

In FIG. 16, one variation of an elongated catheter or extension member 825 is shown, which is similar to the previous embodiment of FIGS. 12A-12C. The catheter 825 again has a diameter and length suitable for introduction into a targeted portion of the colon 804. In a variation, the working end 826 of the catheter is configured with expandable structures such as two temperature resistant occlusion balloons 828A and 828B, although other variations described below include catheters with multiple occlusion balloons. Such occlusion balloons 828A and 828B can consist of complaint elastomeric materials such as silicone or can be fabricated of non-compliant thin-film structures. The catheter 825 can include independent lumens that allow each balloon 828A and 828B to be inflated or expanded independently. The catheter 825 is introduced under endoscopic vision, typically using an articulating endoscope 830 (FIG. 16), which can be a conventional scope used for colonoscopy, or it can be a single-use endoscope that uses an electronic image sensor. Typically, the distal balloon 828A is inflated first under endosopic viewing at a selected location to serve and as anchor and then the proximal balloon 828B is expanded to provide a treatment space S between the two occlusion balloons 828A and 828B.

As in previously described methods, the delivering step includes the vapor V (FIG. 16) undergoing a vapor-to-liquid phase transition, thereby delivering thermal energy to the targeted colon tissue. In the above-described method, the targeted colon tissue includes at least one of epithelium, basement membrane, lamina propria, muscularis mucosa and submucosa. The method treats or modifies the targeted colon tissue to a depth of less than 0.8 mm, less than 0.6 mm, or less than 0.5 mm. The method introduces the flow of vapor over an interval ranging from 1 second to 1 minute, and typically between 5 seconds and 20 seconds for a selected axial length of a subject's colon, which is described below. The vapor generator 840A and controller 840B are configured to deliver energy from the vapor in the range of 10 calories/second to 100 calories/second and in one variation is between 20 and 50 calories/second. In general, a method of treating constipation of a patient comprises delivering vapor into the interior or lumen of the patient's a colon to heat targeted colon tissue and causing a reduction in fluid absorption by or through the targeted colon tissue. The result of such energy delivery consists of modifying, damaging, constricting or otherwise closing fluid absorption pathways in the targeted colon tissue, which thereby treats and reduces constipation. The thin layer ablation of a portion of such a mucosal layer also can modify hormonal signaling from the targeted region, modify secretions from the targeted region, and modify or ablate the microbiome, which is well understood as playing a substantial role in many intestinal disorders.

As can be seen in FIGS. 16 and 17, the expandable structures or balloons 828A and 828B are spaced-apart a selected distance which can be from 5 cm to 40 cm when using two balloons and typically is from about 2 cm to 15 cm. The vapor is delivered from one or more vapor outlets or exit ports 842 in the catheter shaft portion 844 intermediate the balloons 828A and 828B. In a variation, there is an outflow channel in the catheter shaft 825 (not shown) which extends to the exterior of the patient for releasing heated flowable media (air and water droplets) from the treatment site S. In such a variation, there may be a check valve in such an outflow channel (not shown). Typically, the vapor-to-liquid transition cannot over pressurize lumen as the inflow volume of a high-quality vapor collapses into a few drops of liquid. In any event, the system is adapted to maintain a low pressure in the treatment site S of the colon. In other variations, the vapor delivery can be pulsed with ON intervals ranging from 0.01 seconds to 5 seconds and OFF intervals ranging from 0.01 seconds to 1 second.

In general, a method of applying energy to colon tissue to treat constipation or another disorder of the colon comprises generating a flow of vapor and introducing the flow of vapor into an interior of a colon wherein the vapor delivers thermal energy sufficient to modify colon tissue. The flow of vapor can be generated by at least one of resistive heating means, radiofrequency energy means, microwave energy means, photonic energy means, inductive heating means, and ultrasonic energy means. The step of modifying colon tissue includes at least one of causing damage, ablation, sealing, or remodeling of colon tissue. The targeted colon tissue includes at least one of epithelium, basement membrane, lamina propria, muscularis mucosa and submucosa. The flow of vapor can be provided over an interval ranging from 1 second to 1 minute and the flow of vapor can be generated from or at least one of water, saline, alcohol, or a combination thereof. Another step of the method comprises applying negative pressure to the targeted lumen after terminating the flow of vapor. Yet another step comprises introducing a cooling fluid into the colon between the expansion structures (not shown).

In another variation, the method further introduces the flow of vapor with at least one substance or active agent with the vapor, or before or after the vapor. The agent can consist of an anesthetic, an antibiotic, a toxin, a sclerosing agent, alcohol, or an ablation enhancing media.

Referring to FIG. 17, the working end of the catheter 825 are shown with a partially cutaway view. It can be seen that the catheter shaft includes a central vapor delivery lumen 845 that can be that extends distally to at least one vapor exit port position between the first and second occlusion balloons 828A and 828B. In use, it can be seen that the catheter 825 can be introduced through the working channel 832 of an endoscope 830 such that the viewing mechanism can observe the expansion of each of the occlusion balloons 828A and 828B. For example, the endoscope of FIG. 16 can consist of an image sensor 835 with field of view FOV carried at the distal end of an endoscope, which may be a single-use endoscope or conventional colonoscope.

As can be further seen in FIG. 17, the catheter shaft 825 has a first inflation lumen 848a communicating with the interior chamber of the distal occlusion balloon 828A and a second inflation lumen 848b for inflating the second or proximal occlusion balloon 828B. Each of the occlusion balloons can be inflated manually with a fluid (a liquid or gas) and typically is inflated with a gas such as air which is preferred over a liquid since a gas is not a heat sink and this prevents the absorption of energy on the surface of a balloon. The sectional view of FIG. 18 shows that the vapor exit ports 842 can be disposed on multiple sides of the catheter 825 intermediate the occlusion balloons 828A and 828B. In another variation, inflation of the occlusion balloons 828A and 828B can be performed by a pressure source and controller which can expand the balloons to a predetermined internal pressure, for example, between 2 and 50 psi, where a plurality of selected pressures are known to each correlate with an known expanded balloon dimension or diameter, and the physician can then visually observe the approximate body lumen diameter and selected corresponding balloon dimension.

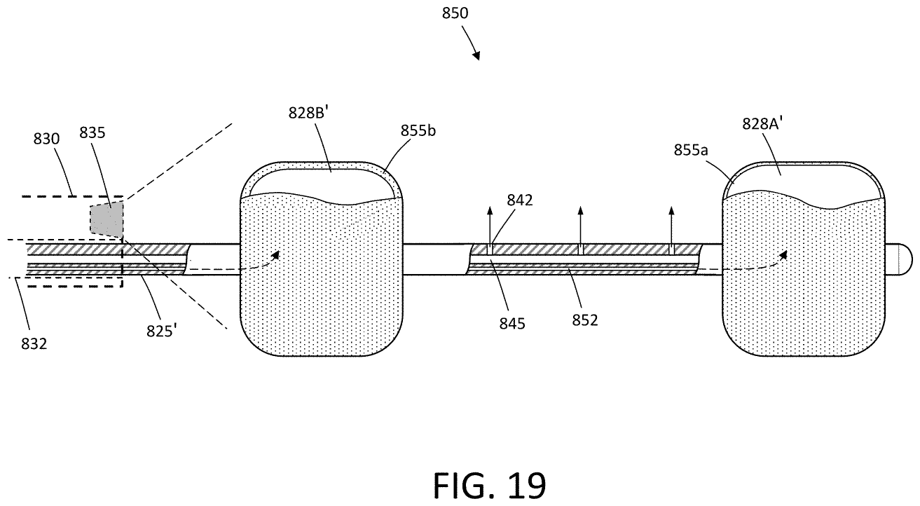

FIG. 19 illustrates a variation of a working end 850 of a catheter shaft 825′ that differs from the previous embodiment that in that a single inflation lumen 852 is provided to inflate both the first and second elastomeric occlusion balloons 828A′ and 828B′. In this variation, the distal occlusion balloon 828A′ has a thin-wall 855a and the proximal occlusion balloon 828B′ has a thicker wall 855b. In this variation, the distal balloon 828A′ will be inflated and expand before expansion of the proximal balloon 828B′ due to the variances in the balloon wall thicknesses. Such a variation in balloon wall thickness allows for endoscopic viewing of the expansion of the distal balloon 828A′ before expansion of the proximal occlusion balloon 828B′ with is useful in allowing observation of the distal balloon expansion at a targeted location as an anchor.

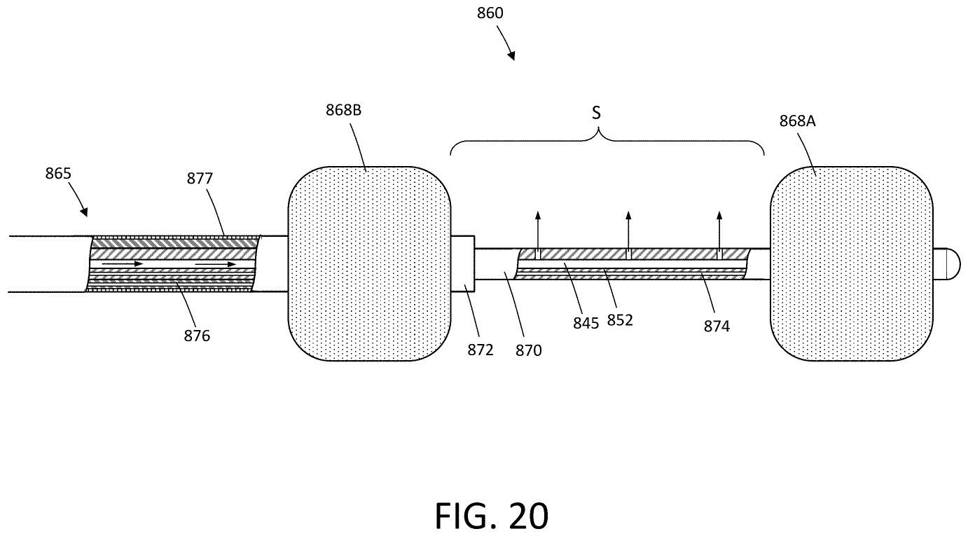

FIG. 20 illustrates another variation of the working end 860 of a treatment catheter 865 with first and second occlusion balloons 868A and 868B that allows for adjustment of the axial spacing between the balloons 868A and 868B to provide different axial lengths of treatment spaces. In particular, the distal occlusion balloon 868A is carried on an inner catheter shaft member 870 that telescopes relative to an outer shaft member 872 that carries the proximal occlusion balloon 868B. By this means, it can be understood that the axial dimension of the treatment space S between the first and second occlusion balloons 868A and 868B can be adjusted from a proximal handle of the catheter. In the variation of FIG. 20, the inner catheter shaft member 870 includes an inflation lumen 874 for inflating the distal occlusion balloon 868A. The outer catheter shaft member 872 includes an inflation lumen 876 for inflating the proximal occlusion balloon 868B. One or more outer insulation layers indicated at 877 can be provided in this variation or other variation herein. The vapor delivery lumen 845 is provided as described previously.

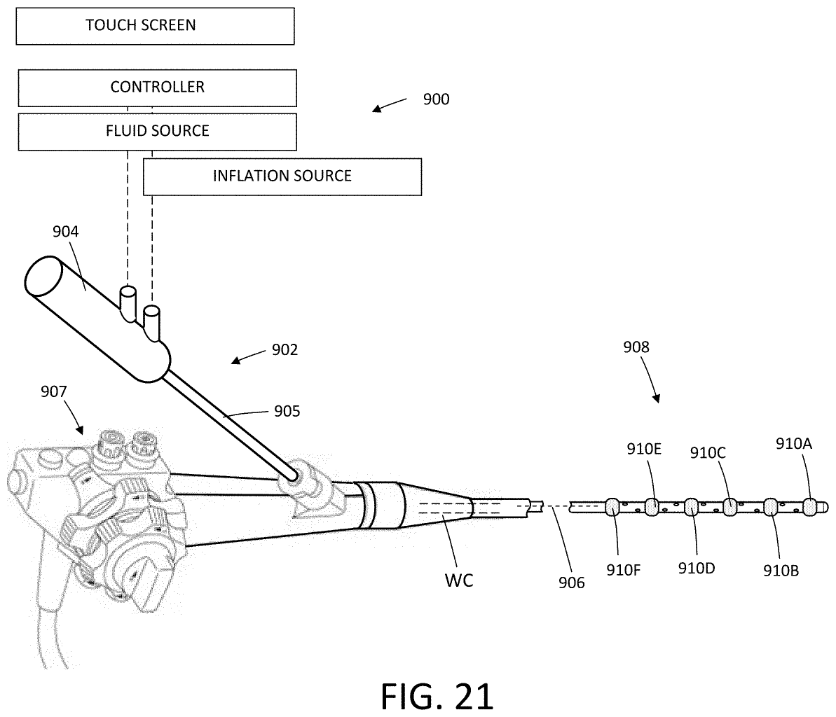

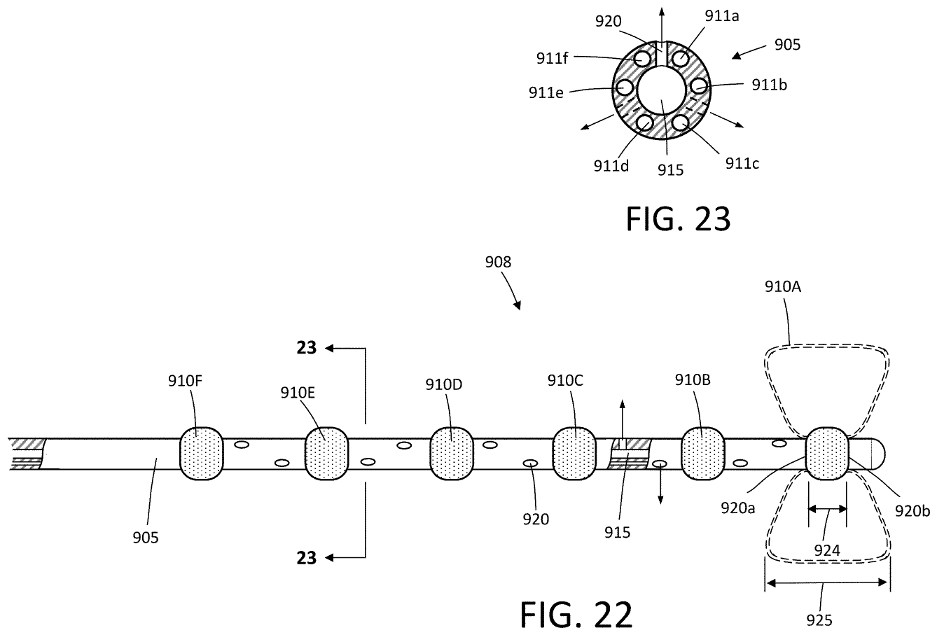

Now turning to FIG. 21, another variation of a treatment system 900 is shown in schematic view where the catheter 902 includes has a handle portion 904 coupled to a catheter shaft 905 extending about central axis 906 that is shown after insertion through a working channel WC of a conventional endoscope or colonoscope 907. As can be seen in FIGS. 21 and 22, the distal region or working end 908 of the catheter shaft 905 carries a plurality of expandable occlusion balloons (shown in non-inflated positions) and in this variation is shown with six such occlusion balloons 910A-910F. FIG. 22 is an enlarged view of the distal region of the catheter of FIG. 21 showing the six occlusion balloons with the distal anchor balloon 910A shown in broken line as when expanded. The sectional view of FIG. 23 shows that the catheter shaft 905 is configured with an independent inflation lumen 911a-911f for each occlusion balloon 910A-910F. The catheter shaft 905 further includes at least one vapor delivery lumen 915 as described previously that communicates with vapor outflow ports 920 positioned between each of the plurality of occlusion balloons.

The catheter system 900 of FIGS. 21-23 and its plurality of axially spaced-apart occlusion balloons 910A-910F are adapted to perform multiple functions. In one variation, the catheter carries six occlusion balloons 910A-910F, however the number of such occlusion balloons can range from 3 to 12°r more. In a first function, the catheter system 900 allows for inflation of a selected pair of occlusion balloons to thereby provide a predetermined treatment space S having a lesser predetermined volume than the catheter 825 of the type shown in FIGS. 16-18 wherein the treatment space was S defined by a fixed axial dimension between two occlusion balloons 828A and 828B. The variation of catheter system 900, shown in FIGS. 21-23, allows for individual treatment sites or spaces of a smaller volume between any selected pair of occlusion balloons, which then also allows for the sequential treatment of adjacent sites between different pairs of such occlusion balloons. In this manner, each treatment site has a reduced volume which allows for a shortened time interval of vapor delivery, which can be advantageous. By performing sequential overlapping treatments, the vapor generating system can be smaller, more economical, and optionally adapted for a single use.

Another function provided by the catheter system 900 of FIGS. 21-23 with multiple occlusion balloons is to allow for effective treatment of body lumens where the targeted treatment site is within a curved or convoluted portion of such a body lumen. In such cases, a single pair of widely spaced-apart occlusion balloons as shown in FIG. 16 may not function. In such a case of a curved or convoluted body lumen, it would be advantageous to treat the lumen wall in short, overlapping segments in sequence.

In one variation shown in FIG. 22, it can be seen that occlusion balloon 910A as well as all other balloons 910B-910F have a proximal end 920a and a distal end 920b in a non-expanded position that extends over a short axial base dimension 924 of the catheter shaft 905. In this variation, each elastomeric balloon is adapted to expand to an expanded shape wherein the radically outward periphery has an axial dimension 925 that is greater than the base dimension 924, which is adapted to engage and provide a seal against the wall of the body lumen. By this means, the greater axial length 925 of each balloon at its peripheral engagement surface allows for gentle engagement of, and sealing against, the wall of the lumen. Also, the regions of the catheter shaft 905 between adjacent occlusion balloons allow for one or more vapor exit or outflow ports 920 between the adjacent occlusion balloons. As will be described further below, the occlusion balloons 910A-910F may be configured with differential wall thickness portions to expand to a desired sectional shape.

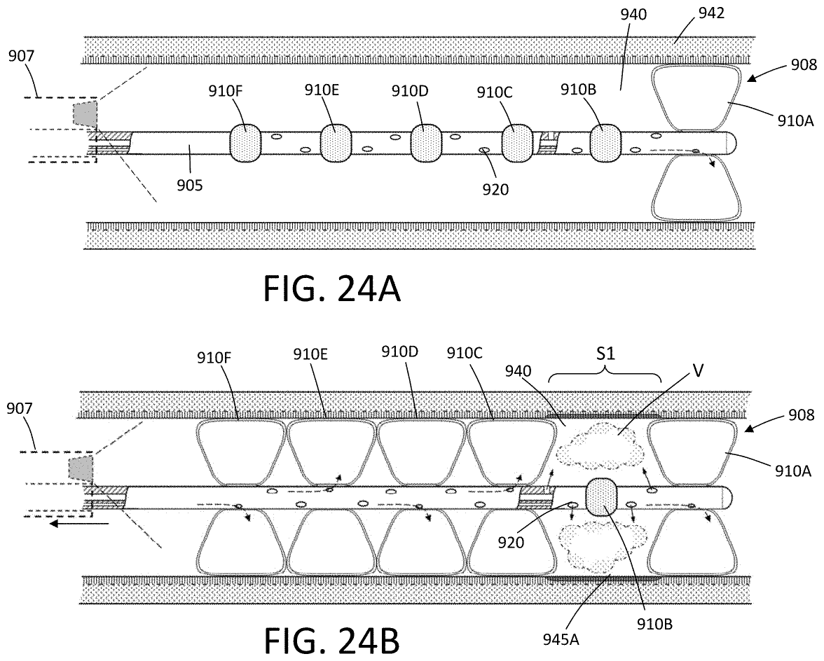

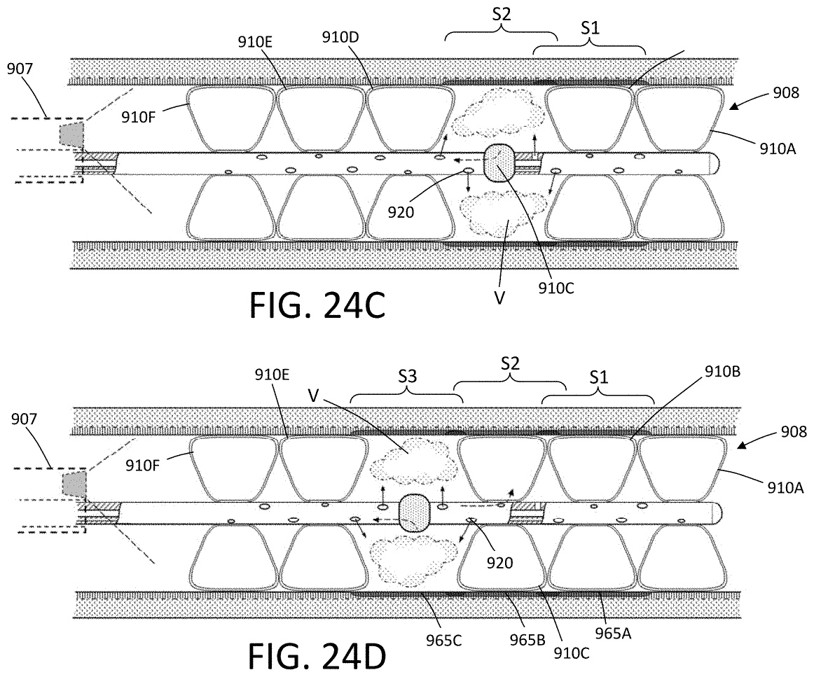

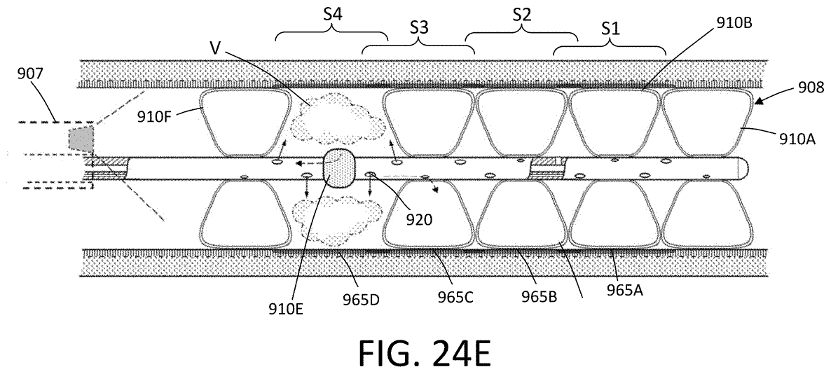

As can be understood from FIG. 23, any pair of occlusion balloons can be expanded to provide a treatment space S therebetween. In one variation of a method, shown in FIGS. 24A to 24E, the physician initially introduced the working end 908 into the body lumen 940 of tubular anatomic structure 942, for example, a subject's colon. The physician inflates and expands the distal occlusion balloon 910A with inflation source 855 as an anchoring balloon under endoscopic vision as described previously to thereby define a distal end of a potential treatment site or space S that extends proximally from such as a distal anchoring balloon 910A. Thereafter, as shown in FIG. 24B, the physician can withdraw the distal end endoscope 907 as needed to better view the inflation of some or all of the other occlusion balloons 910B-910F either sequentially or contemporaneously while leaving the anchoring balloon 910A in its expanded position to thereby define a total potential axial treatment region between balloon 910A and balloon 910F. As can be understood, the physician, or a controller 860A then can actuate the vapor generating mechanism 860B to deliver vapor to exit ports 920 for a predetermined interval to ablate a surface layer of a treatment site between selected pairs of occlusion balloons.

In one method, referring to FIG. 24B, a subsequent step of the method provides occlusion balloon 910B in a non-expanded shape with the balloons 910A and 910C on either side thereof in expanded shapes to thereby define a treatment space indicated at space S1. Thereafter, the vapor generator 860B is actuated to deliver vapor V which ablates a surface layer 965A in 360o around the body lumen 940 in treatment space S1. FIG. 24C show a subsequent step of the method wherein occlusion balloon 910C is deflated and adjacent balloons 910B and 910D are expanded to define an adjacent treatment space S2. Again, the vapor generator 860B is actuated to ablate surface tissue 965B of the lumen 940 in treatment space S2. FIGS. 24D and 24E illustrate a similar steps where spaced-apart occlusion balloons are expanded to ablate surface tissue in treatment spaces S3 and S4 where vapor V is delivered as described above to ablate surfaces 965C and 965D of the body lumen 940 in treatment spaces S3 and S4. By this method, a plurality of adjacent overlapping treatment ablations 965A-965D are performed. As can be understood, the catheter system 900 of FIGS. 21 to 23 can be adapted to treat one or more treatment spaces of various axial lengths to customize the treatment area following introduction of the catheter and anchoring of the catheter with the anchoring balloon 910A. In other words, there is no need to reposition the catheter working end 908 after its initial introduction and anchoring, which is advantageous.

In one variation, the vapor is delivered through the vapor channel 915 and exits the vapor ports 920 between the selected pair of spaced-apart occlusion balloons, while a negligible amount of vapor may leak through vapor ports to a restricted space between adjacent expanded balloons. Such vapor would not reach the wall of the lumen 940 to cause tissue ablation. In another variation, the vapor delivery channel 915 can carry a rotatable interior sleeve (not shown) with a plurality of wall openings therein for aligning with selected vapor exit ports 920, wherein each wall opening aligns with a single set of vapor ports in a treatment space while being out of alignment with all other vapor ports. Thus, by manual or automated rotation of such an inner sleeve, the controller 960A can cause vapor delivery to a single treatment space. In another variation, the catheter shaft 905 can be configured with an individual vapor delivery channel communicating with one or more vapor ports between each pair of spaced part occlusion balloons.

It should be appreciated that a multi-balloon catheter as described above further can be used to ablate tissue in a single treatment space between any pair of occlusion balloon, wherein the controller can calculate the proper treatment interval following the physician's selection of a treatment space, for example, by selection of prompts on a touch screen. In one variation, a catheter for treating Barrett's esophagus or a duodenum may have an oversize balloon used as a stop to abut against a stomach wall at the interface of the body lumen and the stomach as well as an adjacent occlusion balloon that is adapted to contact the wall of the body lumen.

In one variation, the catheter system 900 as shown in FIGS. 21-23 includes a controller 960A that is configured to function robotically to fully automate performance of the steps of the method described above, including inflation and deflation of occlusion balloons 910A-910F by operating an inflation source 955, controlling the vapor generator 960B, which can be carried by the catheter handle, and can also cause flows from a fluid source 960C that delivers liquid to the vapor generator 960B with contemporaneous control of the vapor generator to deliver vapor. In another variation, the vapor generating mechanism 960B can be remote from the handle 904 of the catheter system 900 and controlled by controller 960A. In other words, the system 900 can be configured to operate robotically and perform a customized procedure selected by the physician. For example, the treatment can be planned before or after introduction of catheter working end 908 into the targeted site and expansion of the anchoring balloon 910A. A video touch screen (FIG. 21) with icons can be provided wherein the physician can select (i) the order of inflation of selected pairs of occlusion balloons and thus the treatment space dimensions and volume, (ii) the interval of vapor delivery based on the selection of a lumen diameter and the axial dimension of the treatment space, and targeted ablation depth. In this manner, the entire procedure can be automated after the physician introduces the catheter working end 908 into a targeted body lumen and expands the anchoring balloon 910A.

In general, a method of performing a such a robotic ablation procedure in a subject's body lumen such as a gastrointestinal tract comprises providing a robotic system including: a catheter carrying a plurality of occlusion balloons at a distal end thereof, an inflation source coupled to at least one inflation lumen in the catheter communicating with the occlusion balloons, a vapor generating system communicating with at least one vapor channel in the catheter with at least one vapor exit port in the distal end of the catheter, and a controller adapted to control the inflation source and the vapor generator wherein the steps of the method comprise: introducing the catheter into the subject's body lumen, actuating the controller to sequentially control the inflation source to inflate a selected pair of spaced-apart occlusion balloons to engage walls of the body lumen, actuating the controller to cause the vapor generator to deliver vapor for a selected interval through at least one vapor exit port disposed between the selected pair of occlusion balloons thereby applying ablative energy to a first portion of the walls of the body lumen, followed by inflating at least one other pair of spaced-apart occlusion balloons and repeating the vapor delivery step.

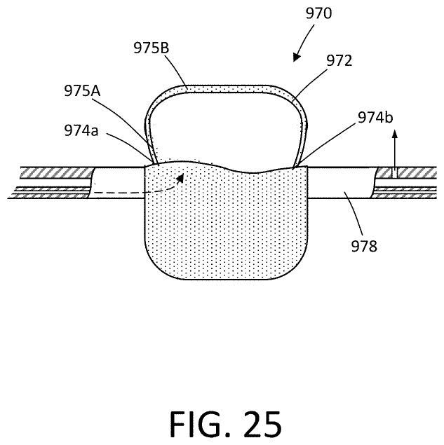

Now turning to FIG. 25, the cut-away view of an occlusion balloon 970 shows that the elastomeric balloon wall 972 has a differing thickness to thereby control the expanded shape of the balloon. Various configurations can be used to thereby control the shape of the balloon walls where ends 974a and 974b of the balloon wall 972 have a first thinner thickness 975A compared to a central portion 975B of the balloon, which is the radially outward portion when the balloon is expanded. In another variation, the balloon wall end portions fixed to the catheter shaft 978 are thicker than the central portion, which can be suited for a gentler engagement with the wall of the body lumen.

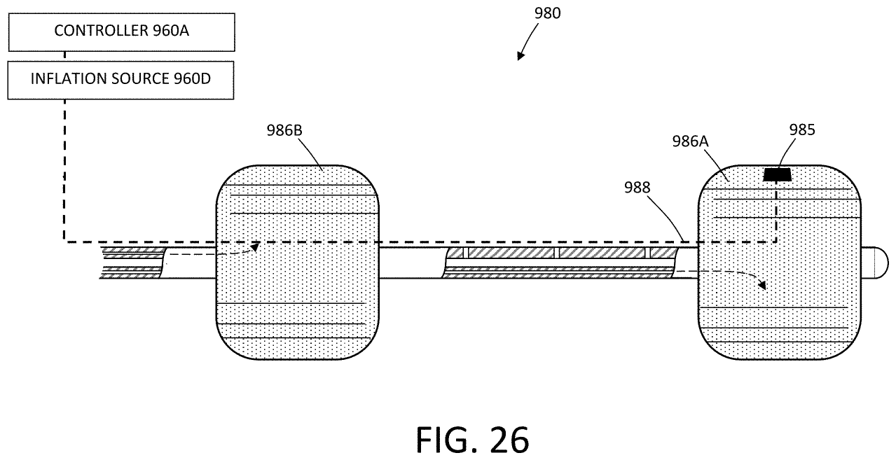

In another variation, shown in FIG. 26, the catheter system 980 can include a sensing mechanism 985 for sensing when one or more occlusion balloons 986A, 966B are expanded to a suitable dimension for contacting and occluding a body lumen. In general, it would be desirable to expand an occlusion balloon 986A to a diameter that somewhat gently contacts a wall of a body lumen to provide a slight seal but does not over-expand the wall of the lumen which could damage the tissue. For example, over-expansion of an occlusion balloon in a patient's esophagus could easily damage tissue which could later result in strictures which would be a serious disorder that might not be correctable, or at a minimum would require surgical procedure to treat such a stricture.

In one variation, at least one occlusion balloon 986A is configured to carry the contact sensor 985 in the surface of the balloon. Wherein the sensor can sense tissue contact and engagement. The sensor 985 would sense contact with the wall of the body lumen and provide a signal to the operator to stop expansion of the balloon 986A. Alternatively, the sensor 985 can send signals to the controller 960A in an automated system that would stop actuation of the inflation source 960D. As shown in FIG. 26, the sensing mechanism 985 can comprise conductive contact coupled by electrical leads 988 to the controller 960A wherein the controller provides electrical current to the sensor and measures capacitance which will easily provide signals of whether the contact sensor is in contact with tissue or not in contact with tissue. In other variations, the controller can measure at least one capacitance, impedance, and phase angle. In an embodiment, an occlusion balloon can carry a plurality of such sensors, from which signal can be compared to ensure that all sides of the occlusion balloon are in similar contact. In a variation, such a sensor carried in single occlusion balloon can be used, and the controller can record the inflation volume in the occlusion balloon carrying the sensor 985, and then the controller 960A can expand the other occlusion balloon with a similar volume of the fluid media to achieve the same diameter in other occlusion balloons. In another variation, each occlusion balloon can carry at least one such sensing mechanism 985.

In general, a system of the invention comprises an elongated catheter configured for insertion into a subject's body lumen, at least one occlusion balloon carried at a distal region of the catheter, an inflation source for inflating the at least one occlusion balloon, a sensing mechanism for sensing a parameter of contact between an inflated occlusion balloon and a wall of the body lumen, a controller adapted to receive signals from the sensing mechanism, and an energy emitter disposed in the distal region of the catheter. The controller is configured to provide an alert when the sensing mechanism senses a predetermined parameter of contact between an inflated occlusion balloon and the wall of the body lumen. In a variation, the controller is operatively coupled to the inflation source, wherein the controller is configured to control the inflation source in response to the signals from the sensing mechanism. The sensing mechanism is an electrical sensor adapted to sense at least one of capacitance, impedance, and phase angle.

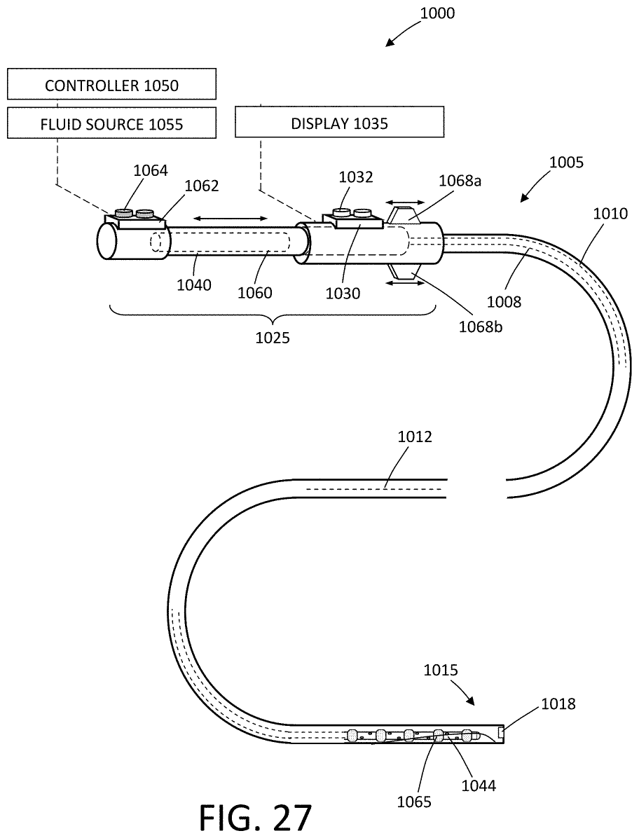

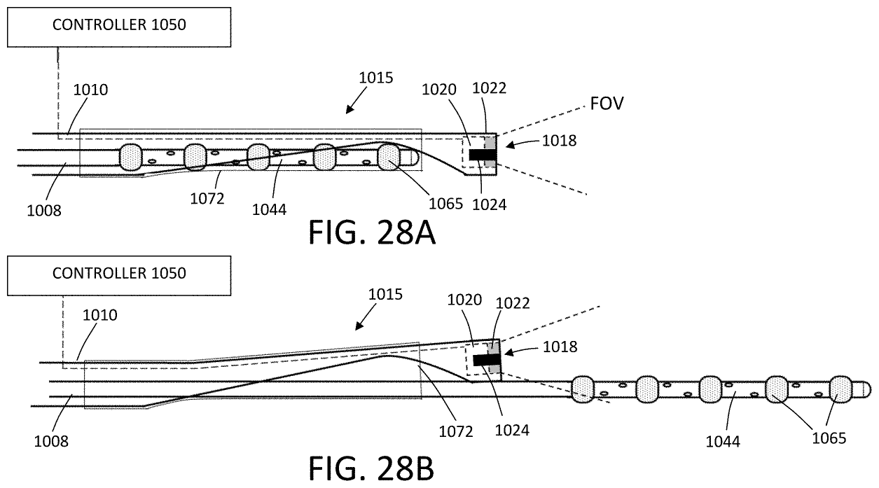

FIG. 27 illustrates another variation comprising a vapor treatment system 1000, which includes a single-use probe 1005 that has an endoscopic viewing component integrated therein together with a catheter 1008 that functions as described previously. Thus, such a single use system 1000 can be used to safely ablate a targeted region in a body lumen without the risk of using conventional endoscopes that are known to be difficult to sterilize. The targeted body lumen can be any lumen in a human or mammalian body and is described in a non-limiting manner herein as a treatment site in a subject's gastrointestinal tract. As can be seen in FIG. 27, the probe 1005 can have a flexible elongated shaft portion 1010 with central axis 1012 that has a length and diameter suited for treating a patient's esophagus, stomach, intestinal region, including the duodenum, as well as the colon. In one embodiment, the probe's elongated shaft 1010 has a diameter of less than 8 mm, less than 6 mm or less than 5 mm. The distal end 1015 of the probe carries an image sensor assembly 1018, which includes a CMOS sensor 1020 and lens 1022 with field of view FOV (see FIG. 28A). The image sensor 1018 is connected to a controller, image processor and display as is known in the art. At least one light emitter 1024 is provided, which typically consists of one or more LEDs but also can consist of light fibers. A handle portion 1025 of the probe 1005 carries a control pad 1030 with one or more actuator buttons 1032 for controlling the imaging system, which can include adjusting light intensity from the light emitter 1024 as well as adjusting operating parameters of the image sensor 1018, which include controlling video, still shots, recording etc. with the image sensor 1018. Images from the image sensor 1018 are displayed on a display 1035. The handle 1025 also includes a manually actuated telescoping member 1040 or similar motor-operated linear drive mechanism adapted to move the distal region 1044 of the catheter 1008 from a retracted portion in the probe to an extended position as illustrated in FIGS. 28A and 28B. In one variation, the telescoping element member 1040 is operatively coupled to a controller 1050 and fluid source 1055, which enables a vapor generating mechanism 1060 within the handle 1025. The handle 1025 can include a second control pad 1062 with actuator buttons 1064 for operating the vapor generating mechanism 1060, which can include an actuator for purging the system and delivering vapor for a preselected time interval as described above.

In one variation, the distal end 1015 of the probe can be articulated with pull wires by the means known in the art, which are operated by articulating grips 1068a and 1068b in the handle 1025. As described previously, all of the control mechanisms in the handle 1025 of the probe 1005 can be automated to provide a fully robotic system.

FIGS. 28A and 28B show one variation of a distal end 1015 of the probe 1005 where FIG. 28A illustrates the probe in an insertion profile in which the probe profile has a small cross-section relative to the outer diameter of the distal region 1044 of the catheter 1008 and occlusion balloons 1065. In FIG. 28B, it can be seen that the distal end 1015 of probe 1005 can be deflected by extension of the catheter 1008. A thin-wall elastomeric sleeve 1072 or tear-away sleeve can be disposed around the distal region 1015 of the probe. By this means, the insertion profile of the probe 1005 can be small and atraumatic. After the distal end 1015 of the probe 1005 is advanced to a treatment site, the profile or cross-section of the probe will increase as the catheter working end or distal region 1044 is deployed. Such a system can be useful, for example, when using naso-gastric access to a patient's esophagus where a small diameter probe is needed. In use, the catheter working end 1044 of FIG. 28B operates as described previously. It should be appreciated that the system of FIGS. 27-28B can be fully automated to function as a robotic system, with the exception of advancing the probe 1005 to a treatment site in a body lumen and inflating the distalmost occlusion balloon 1065 as an anchor.

In general, a single-use system and probe corresponding to the invention for performing a medical procedure in a body lumen of a subject comprises an elongated probe with a central axis configured for insertion into a subject's body lumen, an image sensor positioned at a distal end of the probe, an extendable catheter with a distal region carrying at least one distal occlusion balloon, wherein the catheter is carried in a channel in the probe and wherein said distal region is moveable between a retracted position and an extended position relative to the distal end of the probe, an inflation source for inflating the at least one occlusion balloon, and a vapor generator configured to generate and deliver vapor through at least one vapor port in the distal region of the catheter.

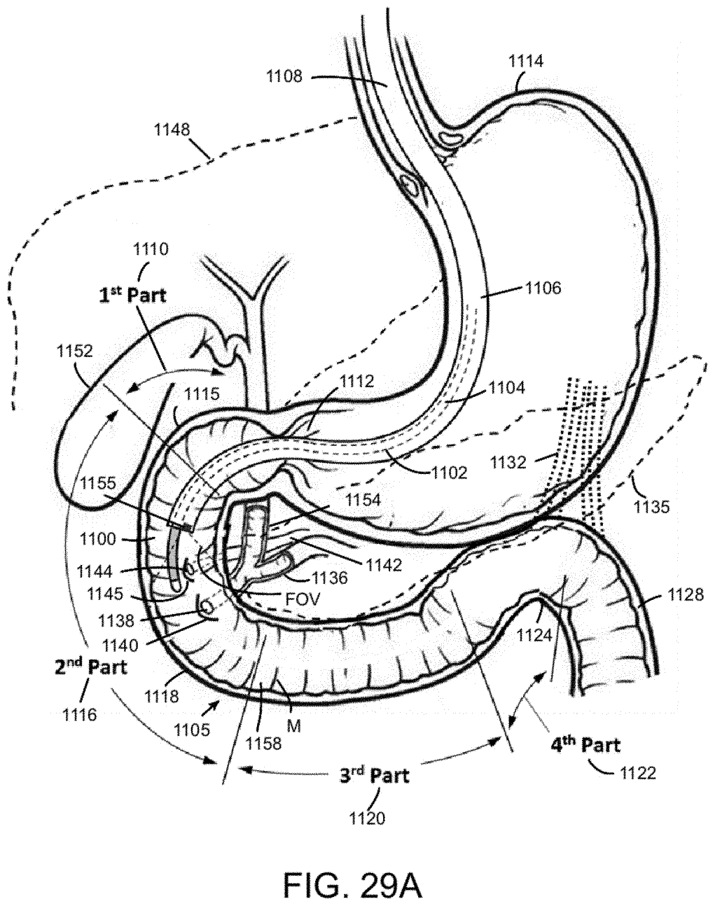

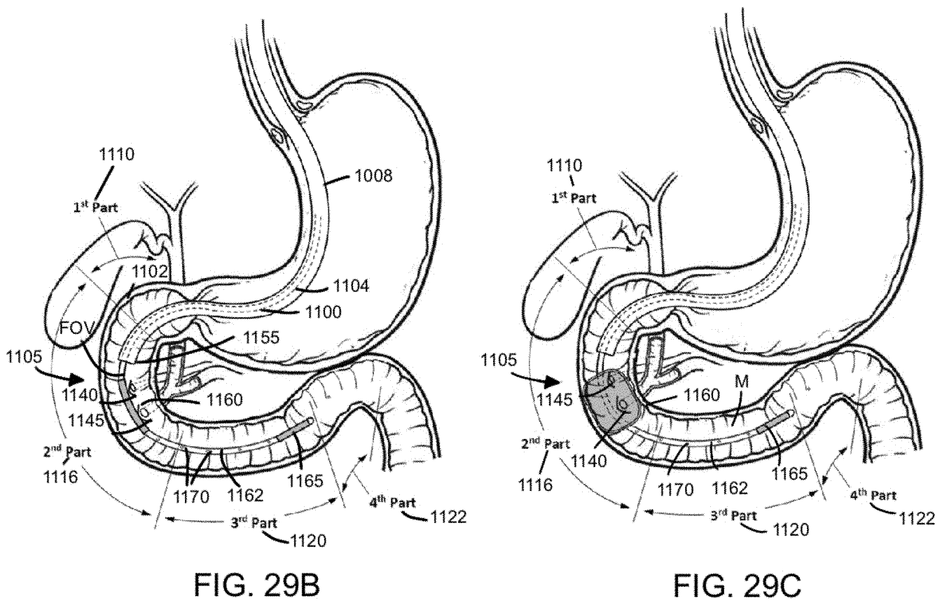

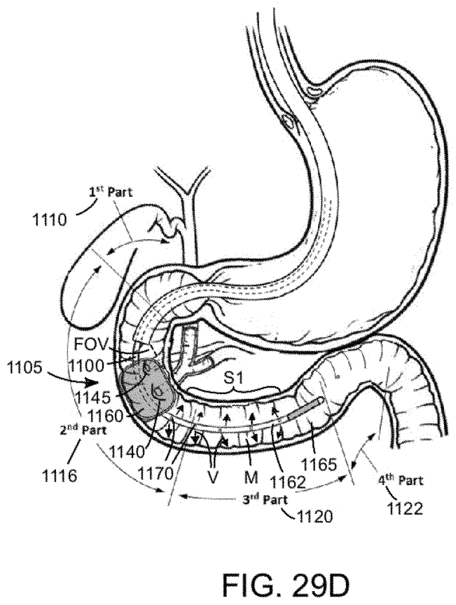

In another variation, referring to FIG. 29A, a probe or catheter 1100 for treating Type 2 diabetes and related metabolic disorders has a catheter shaft 1102 extending about axis 1104 and carries multiple occlusion balloons wherein the catheter is provided with an axial length configured for treating and ablating mucosa M in a targeted site in a patient's duodenum 1105. The catheter typically is configured for introduction through the working channel 1106 of an elongated endoscope 1108 is shown in FIG. 29A. In other variations, a catheter 1100 adapted to apply energy to a patient's duodenum to treat Type 2 diabetes can be a single-use device with an image sensor as described above.

FIG. 29A shows the various parts of the patient's duodenum 1105, which includes the first part 1110 extending away from the pylorus 1112 of the stomach 1114 to the superior duodenal flexure 1115. The first part 1110 of the duodenum 1105 is also called the superior part and typically has a length in the range of 5 cm. The second part 1116 of the duodenum 1105, also called the descending part, extends about 7 to 8 cm downwardly to the inferior duodenal flexure 1118. The third part 1120 of the duodenum 1105 is also called the horizontal part and typically extends about 10 cm. The fourth part 1122 of the duodenum 1105, also called the ascending part, typically has a length of 2 to 4 cm and extends to the duodenojejunal flexure 1124, which transitions to the jejunum 1128. The overall length of the duodenum can range from about 25 cm to 38 cm. The suspensatory ligament 1132 of the duodenum, also called the ligament of Treitz, is coupled to the exterior wall of the duodenum 1105 superior to the duodenojejunal flexure 1124.

Still referring to FIG. 29A, the pancreas 1135 is shown in a dashed line with the main pancreatic duct 1136 extending therefrom to an opening 1138 in the wall of the duodenum 1105 within an anatomic structure called the major duodenal papilla 1140, also known as the papilla of Vater. An accessory pancreatic duct 1142 extends from the pancreas 1135 to a secondary opening 1144 in the wall of the duodenum 1105 within a structure known as the minor duodenal papilla 1145. The major and minor papilla 1140 and 1145 are located in the second part 1116 of the duodenum 1105. FIG. 29A also shows the liver 1148 and gallbladder 1152 with the common bile duct 1154 extending to a junction with the main pancreatic duct 1136.

FIG. 29A illustrates a first step in a method of the invention to ablate duodenal mucosa M using the catheter 1100 to treat Type 2 diabetes, wherein the catheter 1100 is introduced through the working channel 1106 of the endoscope 1108, which has been navigated in a transesophageal approach through the stomach 1114 into the patient's duodenum 1105. The endoscope 1108 has an image sensor 1155 with a field of view FOV that allows viewing of the lumen 1158 of the duodenum 1105. In FIG. 29A, the physician can view the location of the major and minor papilla 1140 and 1145 and can identify and target this location for expanding an occlusion balloon carried by the catheter shaft 1102 to cover and occlude the major papilla 1140 and optionally the minor papilla 1145.

FIG. 29B shows the catheter shaft 1102 being extended distally from the endoscope 1108 within the field of view FOV of the image sensor 1155 until a first occlusion balloon 1160 is in a position adjacent to the major and minor papilla 1140 and 1145. Thereafter, as shown in FIG. 29C, the first occlusion balloon 1160 is expanded or inflated to engage and cover the major and minor papilla 1140 and 1145. The length of the engagement surface of the occlusion balloon 1160 can range from 0.5 cm to 2 cm. In this variation of catheter 1100, a distal region 1162 of the catheter shaft 1102 is disposed between the second or distal occlusion balloon 1165 and the first occlusion balloon 1160. FIG. 29D illustrates the expansion or inflation of the second or distal occlusion balloon 1165 thereby creating a targeted treatment space S1 between the expanded first and second occlusion balloons 1160 and 1165. The axial length of the space between the first and second occlusion balloons 1160 and 1165 can range from 5 cm to 25 cm and typically is between 10 cm and 20 cm.

FIG. 29D further illustrates actuation of the controller a vapor source as described previously to deliver vapor V through vapor ports 1170 in distal region 1162 of the catheter shaft to thereby ablate the mucosa M of the targeted treatment space S1 in the patient's duodenum. In this variation, the mucosa M targeted for treatment is within the third and fourth parts 1120, 1122 the duodenum, and potentially a portion of the second part 1116 distal to the occlusion balloon 1160.

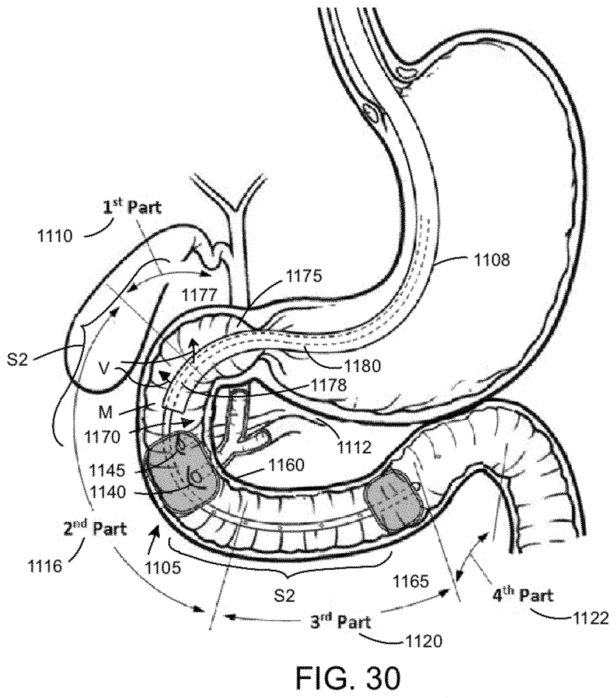

Now turning to FIG. 30, another aspect of the method of treating Type 2 diabetes is shown wherein the catheter 1180 is configured to ablate mucosa M of the patient in the first and second parts (1110, 1116) of the duodenum 1105. As can be seen in FIG. 30, the catheter shaft carries a third or proximal occlusion balloon 1175 that is configured to expand or be inflated in the proximal region 1177 of the first part 1110 the duodenum or potentially in the pylorus 1112. As can be understood, the vapor media V can be delivered through vapor ports 1170 in the catheter shaft region 1178 between the first and third occlusion balloons 1160 and 1175 to ablate the mucosa M in the treatment space S2 therebetween. In this variation, the catheter 1180 typically has a first vapor lumen that extends to the vapor ports 1170 between the first and second occlusion balloons 1160 and 1165 and a second independent vapor lumen that extends to the vapor ports 1170 between the first and third occlusion balloons 1160 and 1175. Thus, the controller and the vapor source can independently and sequentially deliver vapor to the different treatment spaces S1 and S2. In another variation, a single vapor inflow lumen in the catheter shaft can deliver vapor to both the treatment sites S1 and S2 contemporaneously.

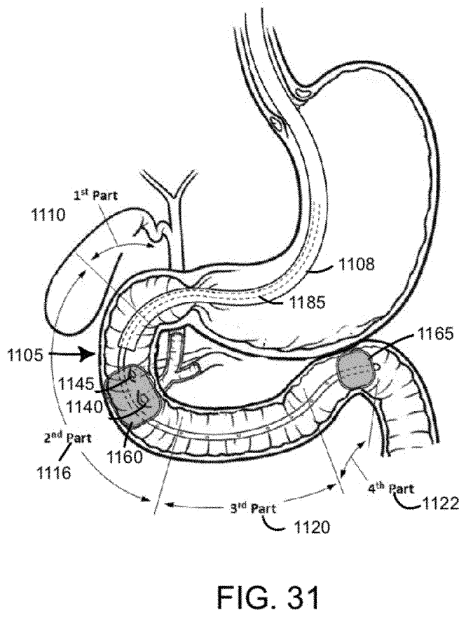

FIG. 31 illustrated another variation of a catheter 1185 where the space between the first and second occlusion balloons 1160 and 1165 has a greater axial length than the variation of FIGS. 29A-29D.

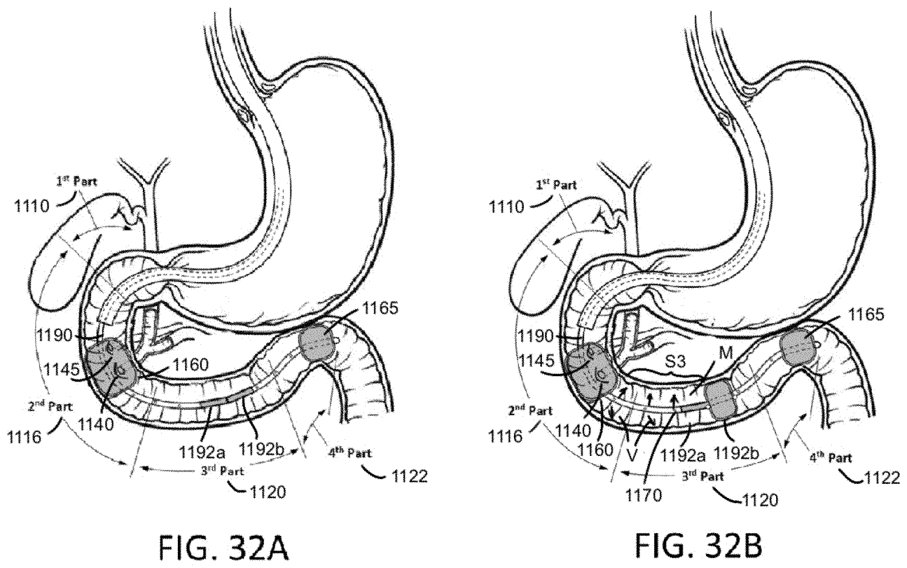

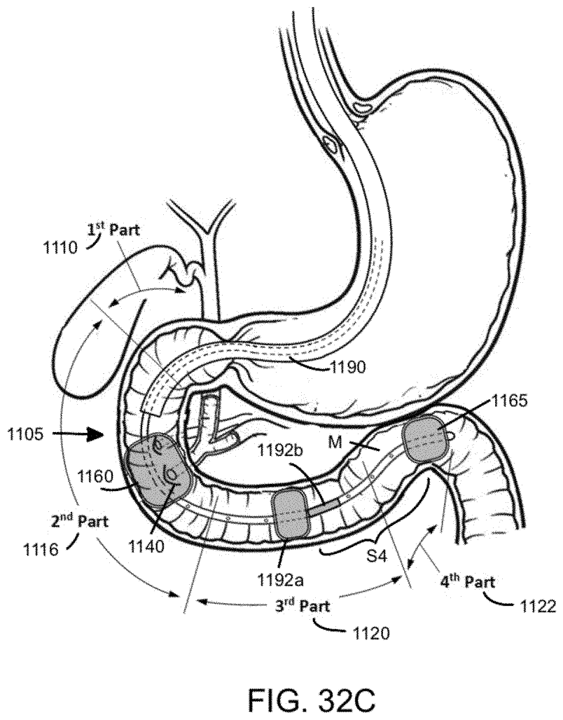

FIGS. 32A-32C illustrate another variation of a catheter 1190 that is similar to the FIG. 31 except that the catheter shaft carries intermediate occlusion balloons 1192a and 1192b that are positioned between the first and second occlusion balloons 1160 and 1165. Such intermediate occlusion balloons 1192a and 1192b are adapted for inflation in the duodenum to split the axial length of the overall treatment space, which may be a preferred method of delivering vapor. As can be seen in FIG. 32B, the occlusion balloon 1192b is inflated to create treatment space S3, and vapor V is delivered through vapor ports 1170 in that space through an independent vapor lumen in the catheter shaft to ablate mucosa M in space S3. Thereafter, FIG. 32C shows the deflation of occlusion balloon 1192b and the inflation of occlusion balloon 1192a followed by a vapor delivery through an independent vapor lumen to ablate mucosa M in space S4.

In another variation, the working end of the catheter can be similar to that of FIG. 20 where the axial space between the first and second occlusion balloons 1160 and 1165 of a catheter as in FIG. 30 can be adjusted by means of the telescoping catheter design of FIG. 20. Similarly, the axial space between the first and third occlusion balloons 1160 and 1175 of FIG. 30 can be adjustable by means of the telescoping catheter design of FIG. 20. In another variation, multiple balloons that are independently inflatable can be provided in the relative positions of the first and third occlusion balloons 1165 and 1175. When using such a catheter, the first occlusion balloon 1160 can be inflated to cover the major papilla 1140 as in FIG. 30, and thereafter a selected occlusion balloon can be inflated in a suitable location to allow for a selected axial length of treatment.

In general, a method of performing an ablation in a subject's duodenum to treat Type 2 diabetes comprises (i) providing a catheter with at least one expandable member at a distal end thereof, (ii) expanding a first expandable member to engage the duodenal mucosa wherein the surface of said expandable member covers the major duodenal papilla, and (iii) applying energy to the duodenal mucosa adjacent the expanded first expanded member to thereby ablate said mucosa.

In a variation, the energy is applied by a flowable media introduced through the catheter to the duodenal mucosa adjacent the expanded expandable member. In a variation, the surface of the expandable member also covers the minor duodenal papilla.

In another variation, a second expandable member spaced-apart from the first expandable member is expanded, and the applied energy ablates a selected length of duodenal mucosa between such first and second expandable members. The energy is applied at a rate of 10 cal/sec to 100 cal/sec and often at a rate of 15 cal/sec to 50 cal/sec. Typically, energy is applied to any treatment space for less than 20 seconds. Typically, a selected length of treatment space is from 5 cm to 25 cm.

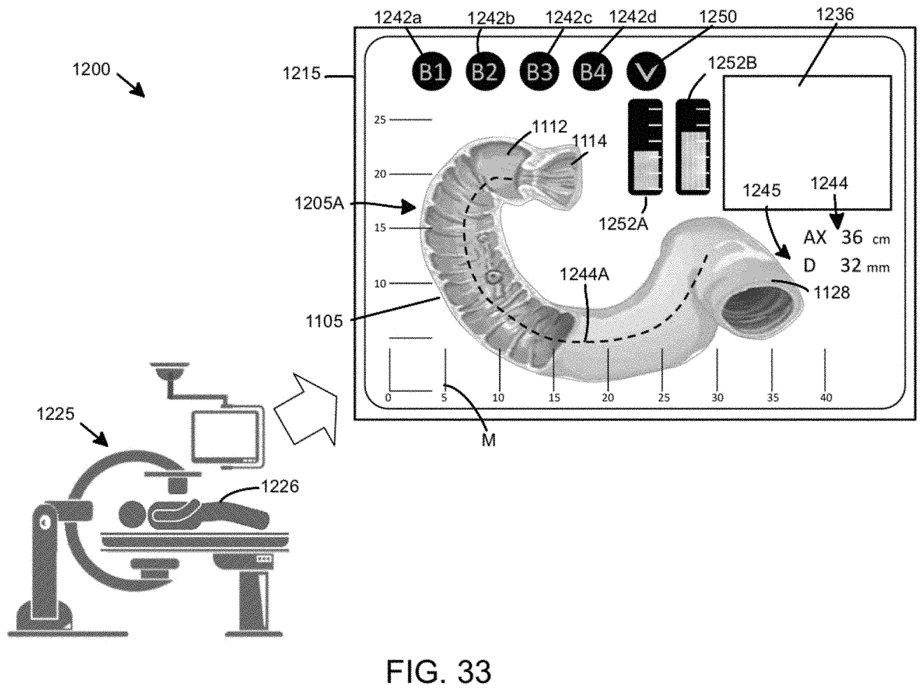

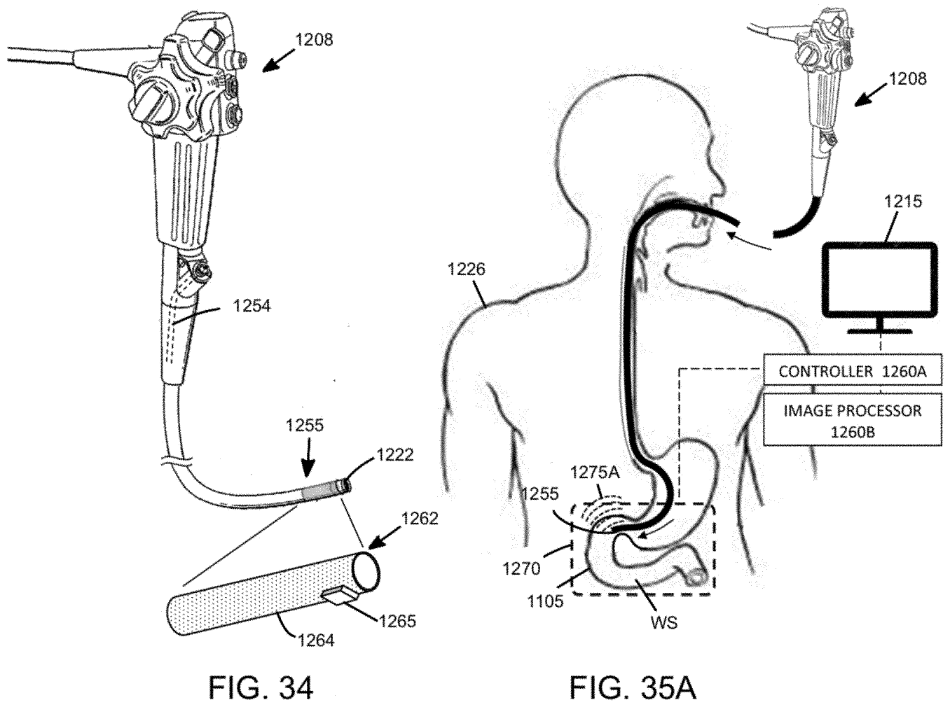

Now referring to FIG. 33, another variation of an imaging and display system 1200 of the invention is shown, which allows the physician to perform a medical procedure in the patient's small intestine by viewing a virtual image 1205A of the patient's intestinal tract and duodenum 1105 in combination with virtual image 1205B of an endoscope 1208 and virtual image 1205C of a treatment catheter 1210 (see FIGS. 34A-34B) in a display screen 1215. Such virtual images 1205A, 1205B and 1205C on the display screen 1215 are adapted to be used together with actual captured video images 1220 taken with the forward-looking image sensor 1222 of the endoscope 1208 (see FIG. 34). Further, as will be described below, the medical treatment comprises mucosal ablation in the patient's duodenum 1105 and jejunum 1128 can be performed with manual movement of the endoscope 1208 and treatment catheter 1210 with a partially or completely robotic system adapted to move either or both the endoscope 1208 and treatment catheter 1210.

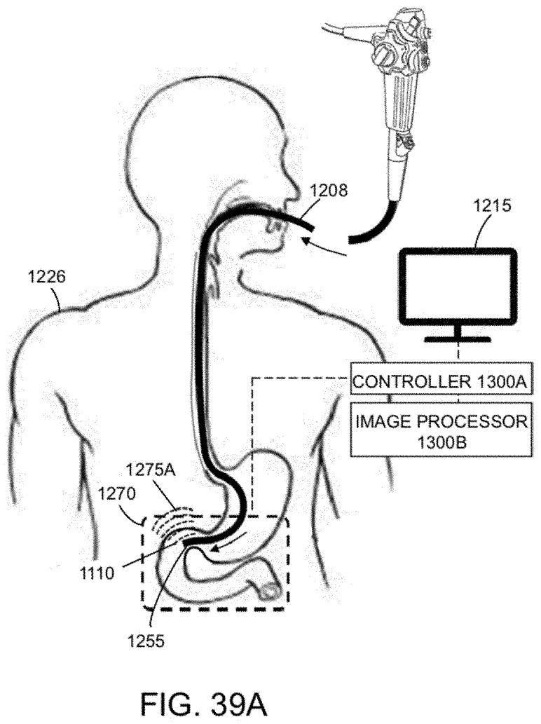

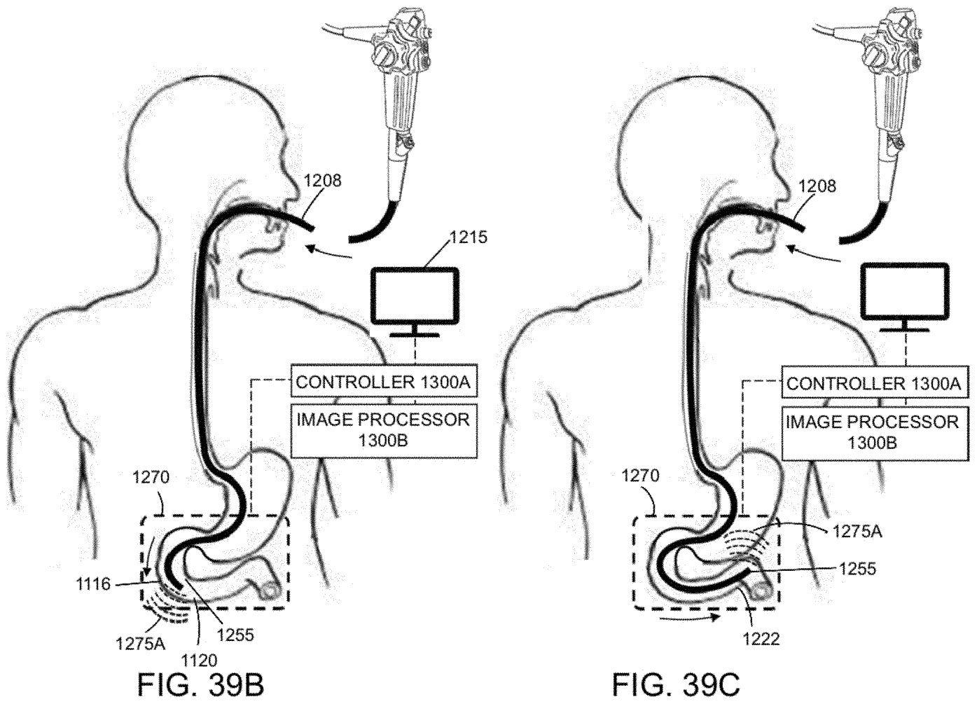

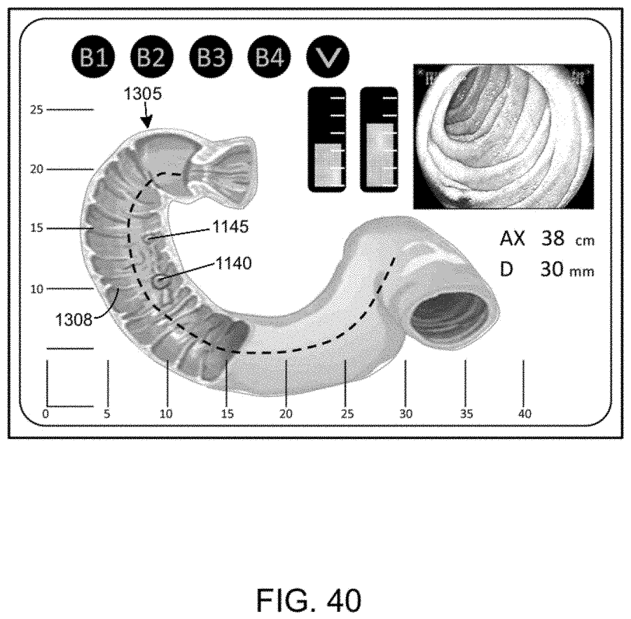

In FIG. 33, the imaging and display system 1200 can include a computed tomography (CT) scanner 1225 that is used to image the patient's body 1226 and thereafter create the virtual image 1205A of a portion of the patient's gastrointestinal tract, typically consisting of the pylorus 1112 and a small part of stomach 1114, the duodenum 1105 and the jejunum 1128 on the display screen 1215. Such a virtual image 1205A can be generated by a CT scanner 1225 which has have been developed in recent years with technological advances, such as allowing for volumetric isotropic voxel imaging and associated improvements in image processing software that allows for advanced virtual images as well as three-dimensional (3-D) visualization. Thus, virtual imaging has become a tool that physicians can use to help plan an interventional procedure, such as a duodenal mucosal ablation procedure as described herein. It should be appreciated that one or more other imaging systems can be used to create the virtual images of the patient's small intestine depicted in FIG. 33, such as magnetic resonance imaging (MRI), ultrasonography, positron emission tomography (PET) and x-ray imaging. Further, a virtual image 1205A of a duodenum as shown in FIG. 33 also may be developed from video capture during direct endoscopic viewing as will be described below in connection to FIGS. 39A-39C and FIG. 40.

Still referring to FIG. 33, the virtual image 1205A of patient's stomach 1114, duodenum 1105 and jejunum 1128 is shown. The virtual image 1205A can be translucent, transparent or displayed as a partially or fully longitudinal sectional view, any of which virtual images can be generated by image processing software. In the variation of FIG. 33, the display screen 1215 includes other features that can be observed by the physician to assist in performing a mucosal ablation procedure. As will be described further below, advancement of the endoscope 1208 and treatment catheter 1210 in the duodenum 1105 will be displayed as virtual images 1205B and 1205C on the display screen 1215 super-imposed over the virtual image 1205A of the patient's intestinal tract.

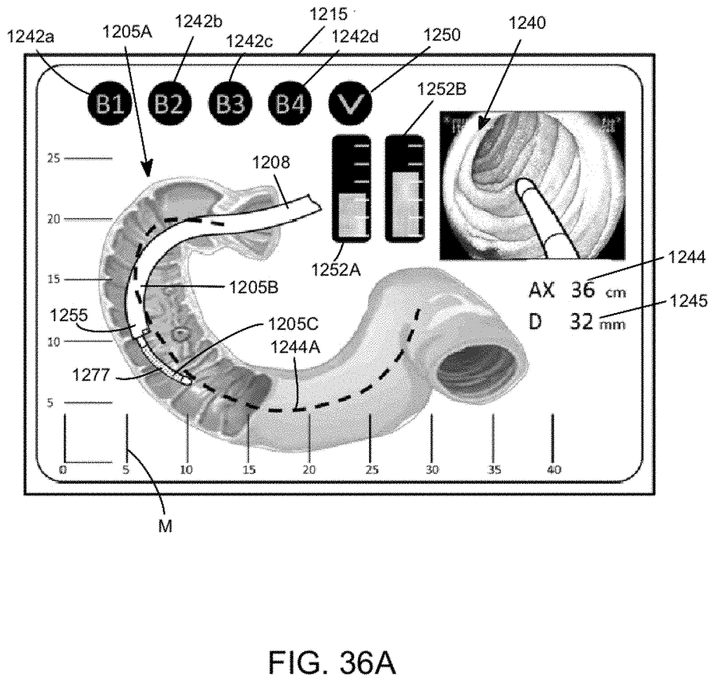

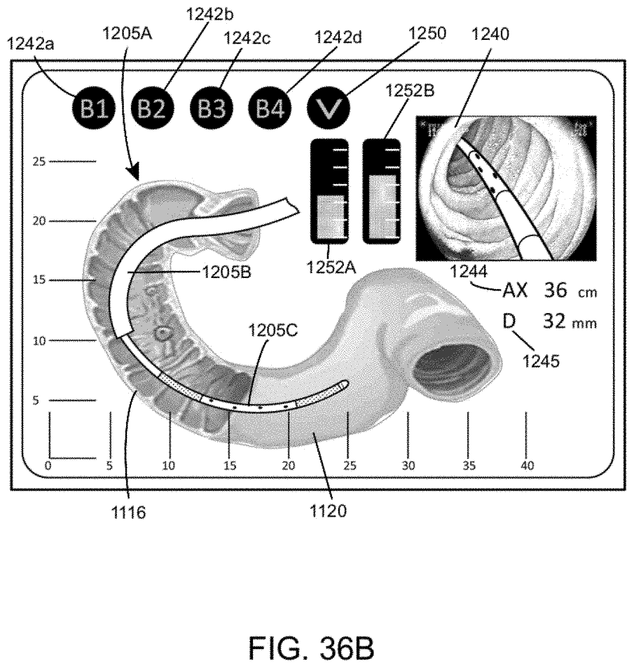

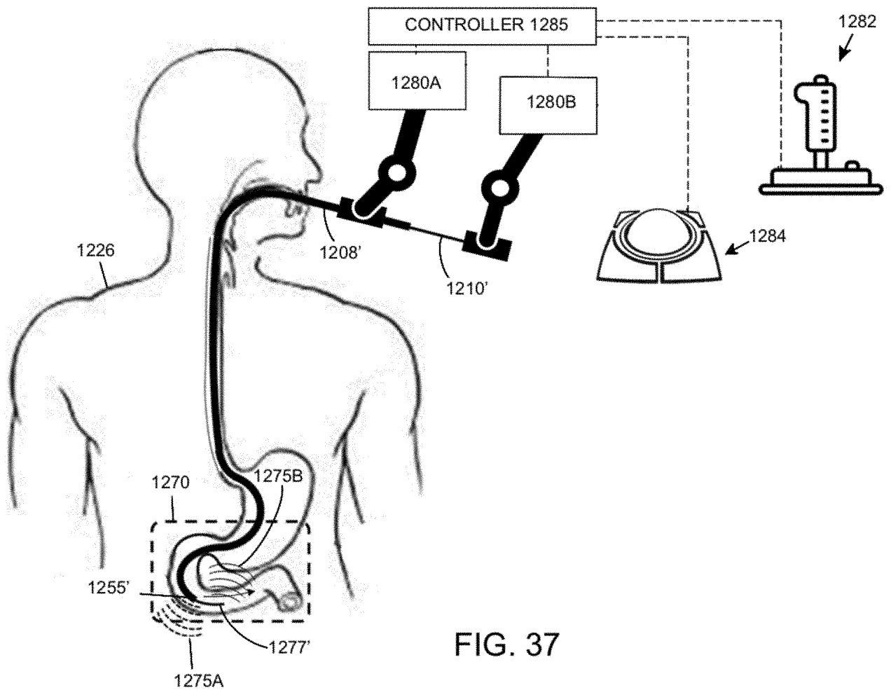

As can be seen in FIG. 33, the upper right quadrant of the display screen 1215 includes a window 1236 in which an actual image or video capture 1240 (see FIG. 36A) from the image sensor 1222 (FIG. 34) of the endoscope 1208 can be displayed. Thus, the physician can perform the medical procedure by referring to both the virtual image 1205A of the duodenum and the actual image 1240 of the intraluminal view of the duodenum 1105.

In FIG. 33, the display screen 1215 carries a number of other indicators and optional controls that can be used to adjust operating parameters of the system and/or observe operative conditions during the medical treatment with the treatment catheter 1210. In some variations, the display screen 1215 functions as a touch screen which can then be used to adjust any operating parameters by touching the display screen 1215. An example of features provided in the display screen 1215, the indicators 1242a, 1242b, 1242c, and 1242d with respective markings B1, B2, B3, and B4 are adapted to indicate expansion and deflation of four respective occlusion balloons 1165, 1192a, 1192b and 1165 carried by the treatment catheter shaft 1102 of the type shown in FIGS. 32A to 32C. Although four such indicators 1242a-1242d are shown in the display screen 1215 of FIG. 33, any number of indicators can be displayed for any type of treatment catheter carrying from two occlusion balloons to any plurality of occlusion balloons. In one variation, each indicator 1242a-1242d can change color to indicate whether the occlusion balloon is inflated or deflated. Each indicator 1242a-1242d also can provide a visual or audible signal to indicate that an occlusion balloon sensor has sensed the proper balloon expansion to contact the wall of the duodenal lumen. Further, the display screen 1215 can display the axial length 1244 of the duodenum 1105 and a virtual image of the axial centerline 1244A of the duodenum 1105. Such an axial length 1244 of the duodenum superimposed on the virtual image 1205A can assist the physician in treatment planning, wherein the potential axial length and location of mucosal ablation can be considered.

FIG. 33 furthers illustrates that an indicator 1245 is provided which indicates the average or specific diameter of the lumen of the duodenum 1105. In FIG. 33, an average lumen diameter is shown at 1245, but it should be appreciated that multiple diameter dimensions can be shown corresponding to different locations on the duodenum 1105. In the virtual image 1205A of the duodenum 1105 of FIG. 33, the location of the major duodenal papilla 1140 and minor duodenal papilla 1145 can be determined by imaging of the main pancreatic duct 1136 and the accessory pancreatic duct 1142 with the imaging and display system 1200.

Still referring to FIG. 33, another indicator or icon 1250 is provided on the display screen 1215, which indicates the ON or OFF actuation of the energy delivery function of the treatment catheter 1210. For example, the indicator 1250 can indicate the ON/OFF status of vapor delivery from a vapor delivery catheter of the type described above with reference to FIGS. 29A-32C. In one variation, the indicator or icon 1250 comprises a touch screen component so that energy delivery can be actuated ON or OFF by touching the indicator 1250, for example, using a vapor delivery catheter in a treatment system as described herein. In FIG. 33, the display screen also carries first and second bar-type indicators 1252A and 1252B, which may be touch screen sliders, wherein the first indicator 1252A is adapted to indicate (or select) the energy delivery interval, which may be from 5 to 20 seconds for a vapor delivery catheter of the type described herein. The second bar-type indicator 1252B is adapted to indicate (or select) the cal/sec of energy delivered by a vapor treatment catheter, which may be from 10 cal/sec to 100 cal/sec 5 to 20 seconds for a vapor delivery catheter of the type described herein. As can be seen in FIG. 33, the display screen also has dimension markings M along the sides of the image horizontally and vertically as a visual aid to the physician which can assist in treatment planning.

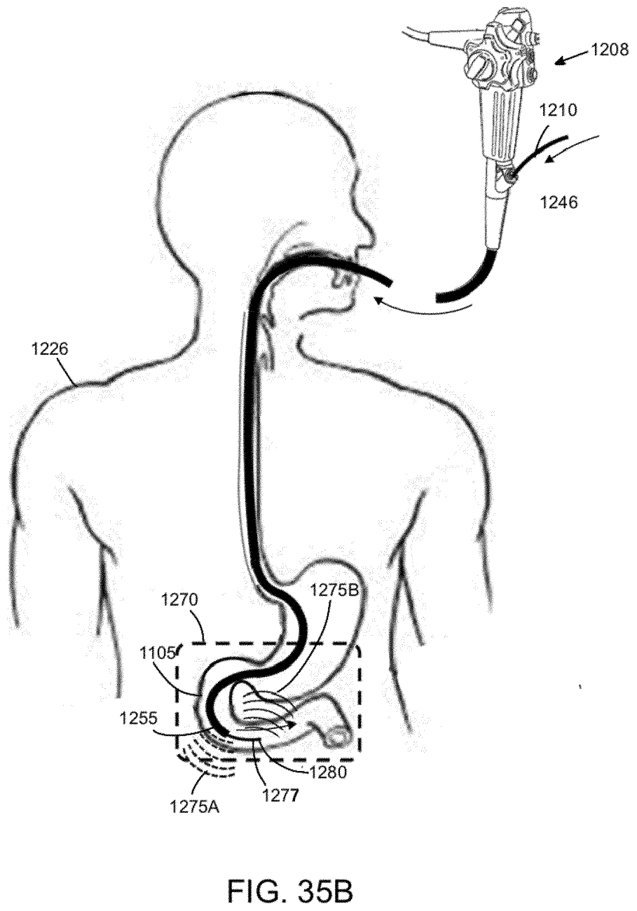

Now referring to FIGS. 34, 35A, and 35B, an endoscope 1208 with a working channel 1254 is shown that can be used in a medical procedure such as a duodenal mucosal ablation as described herein. FIG. 34 shows a conventional, commercially available articulating endoscope 1208 with a distal working end 1255 as is known in art. The endoscope 1208 includes a forward-looking image sensor 1222 that is coupled to a controller 1260A and image processor 1260B as shown in FIG. 35A that provides a real-time capture of actual video images 1240 of the duodenal lumen for display on the display screen 1215 (see FIG. 36A). The endoscope working channel 1254 is typically 3 mm to 6 mm in diameter for introducing the treatment catheter 1210 therethrough into a working space WS in the patient's duodenum 1105.

FIG. 34 also illustrates another component of the invention which comprises a sensor sleeve 1262 configured to be carried by the distal working end 1255 of the endoscope 1208. The sensor sleeve 1262 typically comprises an elastomeric thin-wall tubular member 1264 that carries a sensor component 1265 wherein the assembly or sensor sleeve 1262 is configured for positioning over the distal working end 1255 of the endoscope 1208. The sensor component 1265 can consist of an RFID tag, magnet, coil, passive transponder, or the like that can be sensed by a sensing array 1270, typically an electromagnetic array, at the exterior of the patient's body 1122 as depicted in FIG. 35A. Such a sensor sleeve 1262 typically can be a single-use, disposable device but in another variation can be a re-useable, sterilizable device.

FIG. 35A is a schematic illustration of an endoscope 1208 being introduced into the patient's duodenum 1105, where the sensing array 1270 senses the location of the endoscope working end 1255 and displays its 3-D location on the display screen 1215. More in particular, the sensing array 1270 of FIG. 35A is adapted to respond to the sensor component 1265 carried by the working end 1255 for the endoscope 1208, where signal 1275A is depicted in FIG. 35A. Thus, it can be understood that the sensor component 1265 will respond to an interrogation signal sent by the sensing array 1270, which then can locate the distal working end 1255 of endoscope 1208 in 3-D and map a virtual image 1205B of the endoscope onto the display screen 1215 as shown in FIG. 36A.

FIG. 35B depicts the endoscope 1208 being further advanced into the lumen of the patient's duodenum 1105, where the signal 1275A from the sensor component 1265 is shown communicating with the sensor array 1270. FIG. 35B further shows the introduction of the treatment catheter 1210 through the working channel 1246 of the endoscope 1208 with the catheter's distal end 1277 extending outwardly from the endoscope 1208. In this variation, the distal end 1277 of the catheter 1210 carries a second sensor component 1280 (similar to sensor component 1265) that sends location signals 1275B to the sensing array 1270. Thus, both the endoscope working end 1255 and the catheter distal end 1277 can be spatially located by the sensing array 1277 and thereafter displayed as virtual images 1205B and 1205C on the display screen 1215 as shown in FIG. 36A. In FIG. 36A, it can be seen that the position of the endoscope working end 1255 and the catheter distal end 1277 corresponds to the introduction of the endoscope and catheter as depicted in FIG. 35B. FIG. 36B is another view of the display 1215 similar to that of FIG. 36A showing further advancement of catheter distal end 1277 from the endoscope 1208 while the endoscope distal end is maintained in a stable position.

As can be seen in FIGS. 36A and 36B, the actual image 1240 of the duodenal lumen is shown in the upper right quadrant of the screen display 1215. In FIG. 36A, the distal end 1277 of the treatment catheter 1208 extends slightly into the endoscope's field of view in the actual image 1240. In FIG. 35B, the distal end 1277 of the treatment catheter 1208 extends through the second part 1116 and third part 1120 of the duodenum 1105. The virtual image 1205B the endoscope working end 1255 and the virtual image 1205C of catheter distal end 1277 are superimposed on the virtual image 1205A of the duodenum 1105 and intestinal tract. Thus, it can be seen in FIGS. 36A and 36B that the medical procedure can be monitored and controlled by observation of the virtual images 1205A, 1205B and 1205C and the actual video capture image 1240 to assist in performing the mucosal ablation procedure exactly as planned.

In FIGS. 35A and 35B, it can be understood that the physician manually introduces the endoscope 1208 into the patient's duodenum 1105 and thereafter advances the working end 1277 of the catheter 1210 outwardly from the endoscope into the duodenal lumen. FIG. 37 illustrates an alternative robotic system wherein a first robotic subsystem 1280A is adapted to move the endoscope 1208′ and working end 1255′ and a second robotic subsystem 1280B is configured to advance the treatment catheter 1210′ and distal end 1277′ through a working channel in the endoscope 1208′. The first robotic subsystem 1280A is shown being operated by a joystick 1282 coupled to a controller 1285 wherein the joystick 1282 can be operated by the physician while observing the virtual images 1205A, 1205B, and 1205C on a screen display 1215 and while viewing the actual image capture 1240 as illustrates in FIGS. 36A and 36B. In FIG. 37, the second robotic subsystem 1280B is shown being operated by a rollerball 1284 coupled to the controller 1285 as is known in the art. It should be appreciated any form of joystick, rollerball, or other similar mechanism can be used to operate the robotic subsystems 1285A and 1285B. In all other respects, the imaging system and sensing array 1270 functions as described previously.

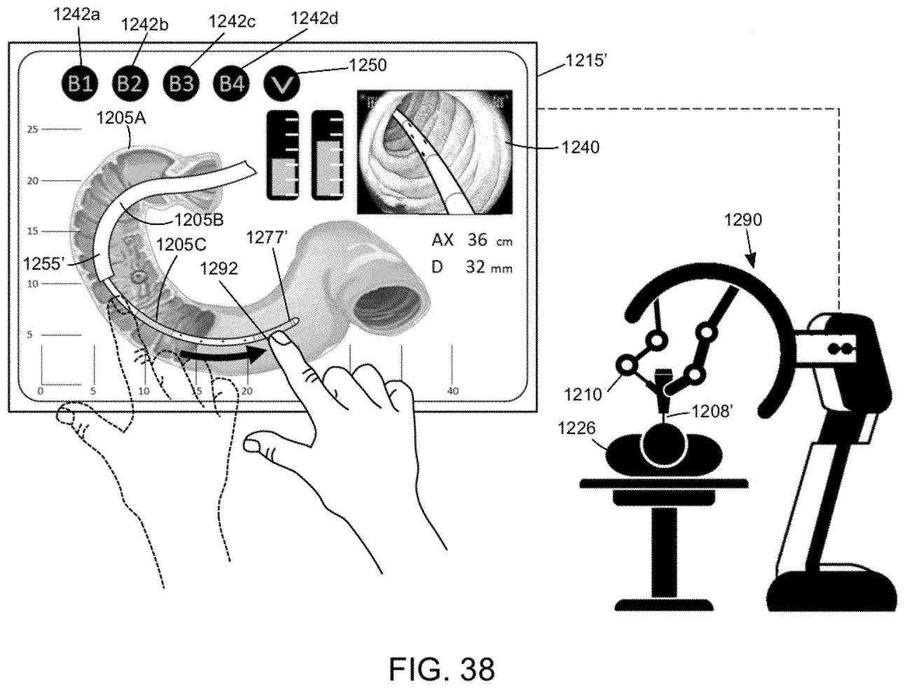

FIG. 38 illustrates another variation of a robotic system 1290 with a touch screen display 1215′ for operating the system. The touch screen display 1215′ is configured to display the virtual images 1205A, 1205B and 1205C as described above together with the actual video images 1240 captured with an endoscope 1208 as described above. In this variation, the physician can simply touch the touch screen display 1215′ and drag a finger 1292 across the touch screen to robotically move the endoscope working end 1255′ and the catheter distal end 1277′ in the duodenum 1105, which are shown as virtual images 1205B and 1205C in FIG. 38. The robotic system 1290 of FIG. 38 also allows for touch screen actuation of inflation/deflation of the occlusion balloons with indicators 1242a-1242d and for actuation of energy delivery with indicator 1250.

As described previously with reference to FIG. 33, the imaging system 1200, for example, a CT system, is performed prior to a mucosal ablation procedure, wherein the imaging system 1200 and image processing software generates the virtual image 1205A of the patient's duodenum on the display screen 1215 as described previously. FIGS. 39A-39C schematically illustrate another method for creating a virtual image 1205′ on a display screen 1215′ shown in FIG. 40. This method does not require the use of CT scanner system 1225 as shown in FIG. 33, which can make a duodenal mucosa ablation procedure more efficient.

In FIG. 39A, it can be seen that the physician introduces an endoscope 1208 as described above into the first part 1110 of the duodenum 1105 of the patient 1226 and the sensor component 1265 carried by the endoscope provides a signal 1275A to the sensor system 1270 as described above which provides a dimensional location of working end 1255 of the endoscope 1208. FIG. 39B illustrates the endoscope working end 1255 being advanced into the second part 1116 and third part 1120 of the duodenum 1105 wherein the sensor system 1270 again tracks the 3-dimensional location of working end 1255 of the endoscope via signal 1275A. FIG. 39C illustrates the endoscope working end 1255 being advanced into the fourth part 1122 of the duodenum 1105 which again is tracked by the sensor system 1270. After tracking the 3-dimensional location of the endoscope working end 1255, the controller 1300A and virtual image processor 1300B generates the virtual image 1305 of the duodenum 1105 as shown in FIG. 40 together with the axial length of the duodenum for display screen 1215 (FIG. 40). The image processor 1300B can further process the captured video images to generate the diameter of the duodenal lumen for the virtual image 1305. Similarly, the captured video images can be used to generate the virtual image locations of the major papilla 1140 and minor papilla 1145 as depicted in FIG. 40. In another variation, the captured video images can be used to generate virtual images 1308 of the actual plications in the duodenum 1105, and display the duodenum in a longitudinal sectional virtual image, a partial or adjustable sectional virtual image or a translucent virtual image. The use of this method of creating the virtual image 1305 on the display screen 1215 can be advantageous in simplifying the mucosal ablation treatment described above. In all other respects, the treatment method can be performed as described above with a virtual image of endoscope and catheter superimposed on virtual image 1305 of the duodenum as shown in FIG. 40.

In another variation, the distal end of the custom endoscope (not shown) can be provided with at least one 3-axis or 4-axis accelerometer or gyroscope, which can be connected to a controller to map the three-dimensional spatial location of the distal end of such an endoscope. In this system variation, the controller will be able to use the spatial location data to generate a virtual image of the patient's intestinal tract for displaying on the display screen. In another variation, such accelerometer or gyroscope can be carried at the distal end of the sleeve that is fitted over a commercially available endoscope. In this variation, similar accelerometers and/or gyroscopes can be carried at the distal end of a treatment catheter to again allow for spatial location of the catheter working end to create a virtual image thereof on the display screen.

In another variation, a custom endoscope and integrated catheter or combination device (not shown) can be provided that carries an integrated treatment probe or catheter that has a working end that is extendable from the distal end of the combination device. In this variation, an entirely independent catheter adapted to insertion through a working channel is not needed. For example, an extendable catheter component can be extended from 10 cm to 40 cm outwardly from the distal end of the endoscope component. In one variation, the catheter component would carry spaced-apart occlusion balloon as described above, with a fluid source and vapor generator for delivering vapor through the catheter component to exit ports between the expanded occlusion balloons. The integrated endoscope and catheter component can be a single use device, with inflation lumen for expanding the occlusion balloons. The integrated endoscope and catheter component can provide a vapor generator mechanism in either a device handle or in a distal region or the elongated combination device. In use, the combination device would be moved within the patient's duodenum, and the catheter component would expand and deflate the occlusion balloons in multiple locations to sequentially treat multiple regions of the duodenal mucosa.

In another variation, the physician can use the endoscope together with the tool in the working channel to deploy markers in the duodenal lumen which then can be used to map the dimensions of the patient's duodenum and to create a virtual image on a display screen. It should be appreciated that any and all of the above-described methods of tracking the spatial location of an endoscope and treatment catheter can be combined with one another in different variations.

While duodenal mucosal ablation procedures are described herein using vapor delivery from a treatment catheter, it should be appreciated that the virtual imaging systems and robotic systems described herein can be used with any type of treatment catheter adapted for duodenal mucosa ablation. For example, the systems and methods of virtual imaging and robotics can be used ablative treatment catheters that deliver or generate a heated liquid, gas or plasma in one or more balloons that engage the duodenal wall, radio frequency energy delivery by means of various expandable members, microwave devices, light energy devices, cryogenic energy delivery devices or any other energy delivery ablation device known in the art.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

1. A method of performing a medical procedure at a targeted site in a small intestine of a patient, the method comprising:
imaging a patient's duodenum and jejunum with an imaging system;
displaying a virtual image of a duodenum and a jejunum of the patient on a display screen;
introducing an endoscope with an image sensor having a forward-oriented field of view into the patient's duodenum;
utilizing a sensor system to display a location of the endoscope in the virtual image of the patient's duodenum on the display screen;
advancing a treatment tool into the patient's duodenum;
utilizing the sensor system to display the location of the treatment tool in the virtual image of the patient's duodenum on the display screen; and
positioning treatment tool proximate the targeted site and actuating the treatment tool to perform the medical procedure.

2. The method of claim 1 wherein the imaging system includes at least one of magnetic resonance imaging (MRI), computed tomography (CT), ultrasonography, positron emission tomography (PET), x-ray imaging and endoscopic imaging.

3. The method of claim 1 wherein displaying the virtual image comprises displaying at least a portion of each of a stomach, pylorus, duodenum and jejunum on the display screen.

4. The method of claim 3 further comprising displaying a real image from an endoscope image sensor of an endoscope on a display screen.

5. The method of claim 4 further comprising moving the endoscope and a treatment tool in small intestine in response to observing at least one of the virtual image and the real image.

6.-10. (canceled)

11. The method of claim 1 wherein the treatment tool is adapted for movement between a first retracted position within a distal portion of the endoscope and a second extended position extending distally from the endoscope.

12. The method of claim 1 wherein the treatment tool comprises a catheter adapted to be advanced through a working channel in the endoscope.

13. The method of claim 1 wherein the treatment tool includes an energy delivery system for applying energy to a wall of the duodenum.

14.-15. (canceled)

16. The method of claim 13 wherein applying energy comprises at least one of a thermal heating treatment or a cryogenic treatment.

17. The method of claim 13 the energy delivery system provides a heated flowable media to apply energy to the wall of the duodenum.

18. (canceled)

19. The method of claim 17 wherein the heated flowable media applies energy to a mucosa directly or conductively through a balloon wall.

20. The method of claim 17 wherein the heated flowable media is heated in a distal portion of the treatment tool.

21.-24. (canceled)

25. A method of providing a virtual image of a region of a small intestines of a patient, the method comprising:
introducing an endoscope with an image sensor having a forward-oriented field of view into a lumen of a patient's small intestine;
utilizing a sensor system to acquire signals of a three-dimensional location of the endoscope as it is introduced through the lumen; and
processing the signals with a processor to generate a virtual image of the small intestine on a display screen.

26. A method of performing a medical procedure at a targeted site in an intestinal tract of a patient, the method comprising:
introducing an endoscope with an image sensor having a forward-oriented field of view into a lumen of a patient's intestine;
utilizing a sensor system to acquire signals of a three-dimensional location of the endoscope as it is introduced through the lumen;
processing the signals with a processor to display a virtual model of the lumen on a display screen; and
advancing a treatment tool in the lumen;
utilizing the sensor system to display a location of the treatment tool in the virtual model of the lumen on the display screen; and
tracking the location of the treatment tool on the display screen to position the treatment tool at the targeted site to perform the medical procedure at a treatment site.

27. The method of claim 26 further comprising utilizing the sensor system to display the location of the endoscope in the virtual model of the lumen on the display screen.

28. The method of claim 26 wherein a virtual model displays at least a region of a stomach, a pylorus, a duodenum, or a jejunum.

29. The method of claim 26 wherein a virtual model displays at least a region of a first part, a second part, a third part, or a fourth part of a duodenum.

30. The method of claim 26 wherein a virtual model displays a major papilla.

31. The method of claim 26 wherein a virtual model displays a minor papilla.

32.-33. (canceled)

34. The method of claim 26 wherein the image sensor captures images of walls of the lumen and the processor is used to process the images to display an appearance of the walls in the virtual model.

35.-41. (canceled)
